(19) United States
(12) Patent Application Publication
(10) Pub. No.: US 2026/0132094 A1
(43) Pub. Date: May 14, 2026

(54) PROCESS FOR OBTAINING HYDROCARBONS, AND ASSOCIATED PLANT

(71) Applicant: TotalEnergies OneTech, COURBEVOIE (FR)

(72) Inventors: Florent PICARD, SOLAIZE (FR); Delphine MINOUX, FELUY (BE); Walter VERMEIREN, FELUY (FR)

(73) Assignee: TotalEnergies OneTech, COURBEVOIE (FR)

(21) Appl. No.: 18/862,235

(22) PCT Filed: May 5, 2023
(86) PCT No.: PCT/EP2023/061916

§ 371 Date: Nov. 1, 2024

(30) Foreign Application Priority Data
May 6, 2022 (FR) FR2204328

(51) Int. Cl. C07C 1/22; C07C 1/04; C07C 1/12; C07C 1/24

(52) U.S. Cl. CPC C07C 1/22; C07C 1/043; C07C 1/12; C07C 1/24; C07C 2529/83; C07C 2529/86

(57) ABSTRACT

Said process involves the following steps:
(a) converting a C1 to C6 alcohol stream to produce a mixture containing paraffins, olefins, aromatics, and water;
(b) separating the water from the mixture to form a water-depleted mixture;
the water-depleted mixture is separated and/or treated to recover hydrocarbons;

The process includes adding, in step (a) of converting the C1 to C6 alcohol stream, a carbon dioxide-containing stream, and jointly converting the carbon dioxide to carbon monoxide in step (a) of converting the C1-C6 alcohol stream.

### CROSS REFERENCE TO RELATED APPLICATIONS

The present application is a U.S. National Phase Application under 35 U.S.C. § 371 of International Patent Application No. PCT/EP2023/061916 filed May 5, 2023, which claims priority of French Patent Application No. 22 04328 filed May 6, 2022. The entire contents of which are hereby incorporated by reference.

### FIELD OF THE INVENTION

The present invention relates to a hydrocarbon producing process comprising the following steps:
(a) converting a C1 to C6 alcohol stream to produce a mixture containing paraffins, olefins, aromatics, and water;
(b) separating the water from the mixture to form a water-depleted mixture; the water-depleted mixture being separated and/or treated in order to recover hydrocarbons.

The present invention relates, for example, to the field of preparation and use of liquid fuels, in particular of such types as jet fuel or renewable aviation fuels. The present invention also relates to the production of olefins (in particular ethylene, propylene, butenes and pentenes, hexenes), by minimizing the production of paraffins from renewable resources based on the conversion of a C1 to C6 alcohol stream into olefins.

Given the paucity of fossil-based resources and the increasingly alarming environmental concerns, and with the objective in particular of reducing greenhouse gas emissions, it is increasingly sought to replace fossil-based molecules with the use of alternative molecules that have a lower carbon footprint.

Renewable fuels derived from biological matter or originating from carbon dioxide transformed in the presence of decarbonized or electrolytic hydrogen (referred to as “E-fuels”) are an alternative to conventional fossil fuels.

Conventional jet fuels may be blended with bases derived from renewable feedstocks as provided for in the standard D7566-21, thus enabling the production of alternative aviation fuels. Examples of bases for aviation fuel derived from renewable feedstocks that may be incorporated into fossil-based jet fuel are as follows:
synthetic paraffinic kerosenes [SPK], derived from processes such as the Fischer-Tropsch process;
synthetic paraffinic kerosenes produced via the ‘Alcohol-to-Jet’ process (transformation of alcohol into isoparaffinic kerosene) [ATJ-SPK];
synthetic isoparaffins produced by hydrotreating iso-olefin intermediates, produced from fermented sugars [SIP-HFS];
synthetic aromatic kerosenes obtained by alkylation of light aromatics from non-petroleum sources [SPK/A];
synthetic kerosenes obtained from the hydrothermal conversion of fatty acid and fatty acid esters;
paraffinic kerosenes [SPK] obtained from hydrotreated hydrocarbons, esters and fatty acids.

At present, these bases for renewable aviation fuel cannot, for the most part, be used on their own, due to their composition being very far removed from that of fossil fuels. This difference in composition poses problems of compatibility with the materials of elements with which the fuel comes into contact. In this regard, the absence of aromatic compounds in the majority of available renewable aviation fuel bases could lead to problems of compatibility with materials, and in particular with certain seals.

### BACKGROUND

EP2123736 describes a process for producing a diesel fuel using a feedstock in the form of C1 to C5 alcohols, which may be entirely or partially of biogenic origin, wherein, based on a mixture of olefinic hydrocarbons obtained at least partially by dehydration of C1 to C5 alcohols, with a proportion of odd olefins and iso-olefins, a synthetic hydrocarbon is oligomerized or is subjected to hydrogenation. After subsequent hydrogenation and rectification, an aviation fuel is formed with a freezing point of −47° C. or less.

US20210078921 describes the conversion of methanol to gasoline which can be carried out using a heavy gasoline treatment, followed by a separation operation.

U.S. Pat. No. 4,543,435 describes a conversion process for converting an oxygenated feedstock comprising methanol, dimethyl ether or the like to liquid hydrocarbons, the process comprising contacting the feedstock with a zeolite catalyst in a primary catalyst stage at an elevated temperature and moderate pressure in order to convert the feedstock into hydrocarbons that comprise C2-C4 olefins and C5+ hydrocarbons.

EP1844125 relates to a production process whereby synthetic fuels are produced, according to which in a first step, a gaseous mixture comprising methanol and/or dimethyl ether and/or another oxygenated molecule, as well as steam, is converted into olefins having preferably between 2 and 8 carbon atoms, at temperatures of between 30° and 500° C.; and in a second step, the mixture of olefins obtained is oligomerized at higher pressure into higher olefins that contain substantially more than 5, preferably between 10 and 20, carbon atoms. According to this process, a) the production of olefins in the first step is carried out in the presence of a gaseous stream composed essentially of saturated hydrocarbons, which are separated from the product stream of the second step and are returned to the first step; and b) the production of olefins in the second step is carried out in the presence of a stream of water vapor, which is separated from the product stream of the first step of the process and is returned to the first step of the process.

EP2147082 describes a production process for producing synthetic fuels from a mixture, containing hydrogen and oxygenated compounds such as methanol and/or dimethyl ether: during a first step, the mixture is reacted over a catalyst, in order to obtain a hydrocarbon product containing olefins that preferably have 2 to 8 carbon atoms; and, during a second step, the hydrocarbon product thus obtained is oligomerized into long-chain olefins, from which it is possible to obtain products that are gasoline and gas oil.

WO2011061198 describes a hydrocarbon production process for producing hydrocarbons in the form of gasoline, by converting synthesis gas in order to obtain an oxygen-containing compound, such as methanol and/or dimethyl ether, in a first converter; and by further conversion into hydrocarbons in a second converter.

EP2940103 describes a biofuel preparation process for preparing biofuels using ethanol by converting ethanol into a mixture with hydrocarbons, in a catalytic process on a bed of aluminosilicate of such type as zeolite, preferably in the presence of a hydrogen form of the zeolite catalyst.

EP2720990 describes an alcohol conversion process for converting an alcohol into a hydrocarbon, the process comprising contacting said alcohol, as a component of an aqueous solution at a concentration of no more than 20%, with a metal-loaded zeolite catalyst at a temperature of at least 100° C. and up to 550° C., wherein said alcohol may be produced by a fermentation process and is selected from among ethanol, butanol, isobutanol, or a combination thereof; said metal includes vanadium; and said metal-loaded zeolite catalyst is catalytically active so as to convert said alcohol into said hydrocarbon.

EP3795658 describes methods that reduce energy and water consumption in fuel production processes for producing fuel from renewable alcohol-containing feedstocks. The alcohol is converted directly into hydrocarbon transport fuels by a catalytic process, with heat being transferred between intermediate process liquids in order to reduce thermal energy consumption. Overall water consumption is reduced by means of recovering catalytic process water and reducing water temperature which serves to reduce evaporation losses.

US20160090333 describes hydrocarbon production methods for producing aviation range hydrocarbons from biorenewable sources, such as the oligomerization of C3-C8 biorenewable olefins, for example, derived from C3-C8 alcohols produced by the fermentation of biomass. The production of aviation range hydrocarbons is increased by the use of an additional oligomerization zone to oligomerize gasoline separated from the effluent of a primary oligomerization zone in which C3-C8 biorenewable olefins have first been subjected to oligomerization.

WO201145535 describes a distillate production process for producing a distillate from a feedstock of heteroatomic organic compounds comprising at least one heteroatom selected from oxygen, sulfur, halogen, either individually or in combination, wherein the processing of the feedstock comprises at least one conversion step of converting the heteroatomic organic compounds into olefins which is carried out in a first conversion zone; and, in at least one second oligomerization zone, an oligomerization step of oligomerizing olefins originating at least in part from the conversion zone, in the presence of at least 0.5% by mass of oxygenated compounds, in order to produce a distillate. This process enhances the yield of distillate, making it possible to obtain a higher rate of oligomerization as compared with oligomerization of the same feedstock under the same reaction conditions..

WO2022/063994 describes a process for obtaining a jet fuel comprising a conversion step of converting a stream of oxygenated compounds at reduced temperatures (for example lower than 350° C.), with pressure levels of the order of 5 bar to 10 bar and high hourly space velocities (6 h⁻¹ to 10 h⁻¹) followed by a joint oligomerization and hydrogenation step in the same reactor. This process tends to ensure that the ethylene and aromatics produced during conversion are kept at very low content levels.

These processes are not entirely optimized with regard to the conversion of oxygenates in order to produce a mixture of suitable olefins at the conclusion of the conversion.

### SUMMARY

One aim of the invention is therefore to provide a process for obtaining hydrocarbons derived exclusively or at least partially from renewable feedstocks, which is both productive and efficient, particularly in the conversion of feedstocks to olefins.

To this end, the subject matter of the invention is a hydrocarbon producing process of the aforementioned type, characterized by the adding, in conversion step (a) of converting the C1 to C6 alcohol stream, of a carbon dioxide-containing stream, and jointly converting the carbon dioxide to carbon monoxide in conversion step (a) of converting the C1-C6 alcohol stream.

The process according to the invention may comprise one or more of the following characteristic features, taken into consideration individually or in accordance with any technically feasible combination:
the carbon dioxide-containing stream comprises more than 5% by mass of carbon dioxide;
the mass ratio of carbon dioxide to C1-C6 alcohols in the feedstock supplied to conversion step (a) is between 5% and 75%;
at least 2 mol % of the carbon dioxide contained in the carbon dioxide-containing stream is converted into carbon monoxide during conversion step (a) of converting the C1 to C6 alcohol stream;
conversion step (a) is carried out using at least one catalyst comprising molecular sieves containing at least 10 oxygen atom sized pores (10-MR) or larger in their microporous structure;
the catalyst for carrying out conversion step (a) comprises: a phosphorus-modified zeolite, in particular having a P content of at least 0.05% by mass, and preferably between 0.3% by mass and 7% by mass; a composite catalyst comprising at least 0.1% by mass of silicate; or a molecular sieve modified with phosphorus and with an alkaline-earth or rare-earth metal (MP modified molecular sieve), advantageously having an M/P molar ratio in the molecular sieve of less than 1.
the catalyst for carrying out conversion step (a) has been modified by adding one or more metals selected from: Group IIB metals, in particular Zn; Group IIIB metals, in particular Ga; Group VIIIB transition metals, in particular Fe and/or Ni and/or Pt; Group VIB metals, in particular Mo; Group IB metals, in particular Cu and/or Ag; or even metals from the lanthanide group, in particular La;
the catalyst for carrying out conversion step (a) is a phosphorus-modified zeolite having partially an ALPO structure, or is a B-modified zeolite;
the process comprises a separation step of separating the water-depleted mixture into a C1-C2 hydrocarbon fraction, and into a C3+ hydrocarbon fraction, with a portion of the C1-C2 hydrocarbon fraction advantageously being recycled to the conversion step (a) of converting the C1 to C6 alcohol stream;
the carbon dioxide, carbon monoxide and hydrogen present in the C1-C2 hydrocarbon fraction are separated from the hydrocarbons, in particular by cryogenic distillation, membrane separation, or pressure swing adsorption and combinations thereof, with the carbon dioxide, carbon monoxide, and hydrogen then being advantageously recycled to a preliminary synthesis step for synthesizing alcohols intended to form the C1 to C6 alcohol stream, in particular by synthesis gas fermentation and by catalytic conversion of the synthesis gas in order to produce methanol;
the process comprises the following steps:
(c) oligomerizing olefins from the water-depleted mixture;
(d) alkylating aromatics from the water-depleted mixture;
(e) formation of a hydrocarbon stream from at least a portion of the olefins oligomerized in step (c) and at least a portion of the aromatics alkylated in step (d);
the process includes the following steps:
(f) hydrogenation of the hydrocarbon stream formed in step (e) in order to form a hydrogenated hydrocarbon stream;
(g) recovery of at least one jet-fuel fraction from the hydrogenated hydrocarbon stream;
the jet fuel fraction comprises between 2% by volume and 30% by volume of C8+ aromatics, preferably between 8% by volume and 25% by volume of C8+ aromatics;
in the mixture of paraffins, olefins, aromatics, and water produced in the conversion step (a), the ratio of the mass of C3+ olefins to the total mass of olefins is greater than or equal to 0.8;
the C2 to C6 alcohol stream is obtained by fermentation of biomass or synthesis gas, and/or the C1 to C6 alcohol stream is obtained by catalytic conversion of carbohydrates, carbon monoxide or carbon dioxide in the presence of hydrogen;
the hydrogenation step (f) of hydrogenating the hydrocarbon stream to be hydrogenated is carried out separately from and downstream of the oligomerization step (c) of oligomerizing the olefines originating from the water-depleted mixture as well as the alkylation step (d) of alkylating the aromatics originating from the mixture;
the catalyst for carrying out conversion step (a) comprises a zeolite with pore sizing of 10 oxygen atoms or more, modified by the adding of B either before, after, or simultaneously with the formulation step of formulating the final catalyst;
the catalyst for carrying out conversion step (a) has a Si/Al atomic ratio—as measured by chemical analysis, in particular by NMR, taking into account only those Al which form part of the framework structure of the molecular sieve—of between 4 and 500, preferentially between 5 and 200, or more preferentially between 12 and 150;
the process comprises the stripping of at least a portion of the water separated in separation step (b) in order to produce a stream of extracted hydrocarbons, advantageously recycled to separation step (b), and a stream of treated water.

The subject matter of the invention also relates to the use of a jet fuel fraction produced from hydrocarbons obtained by implementing the production process as defined above in the following form:
(i) pure; or
(ii) in a mixture with a jet fuel resulting from the distillation of a petroleum and/or another renewable source,
in order to power at least one aircraft engine.

Advantageously, the jet fuel fraction produced comprises between 2% by mass and 30% by mass of aromatics, in particular between 6% by mass and 20% by mass of aromatics.

In particular, the jet fuel fraction comprises between 2% by mass and 30% by mass of aromatics having at least 8 carbon atoms, in particular between 6% by mass and 20% by mass of aromatics having at least 8 carbon atoms.

Preferably, more than 50% by mass of the aromatics contained in the jet fuel fraction are monoaromatics having 8 to 14 carbon atoms.

It advantageously comprises between 5% by mass and 20% by mass of cycloparaffins and at least 50% by mass of isoparaffins.

The process according to the invention thus makes it possible to obtain a renewable aviation fuel that is totally substitutable (‘drop-in’) or compatible with current engine and fuel systems for aircraft.

The subject matter of the invention also relates to a hydrocarbon producing plant for obtaining hydrocarbons, which comprises:
a conversion stage for converting a C1 to C6 alcohol stream in order to produce a mixture containing paraffins, olefins, aromatics, and water;
a separation stage for separating the water from the mixture in order to form a water-depleted mixture;
at least one separation/treatment stage for separating and/or treating the water-depleted mixture in order to recover hydrocarbons,
characterized in that the conversion stage for converting the C1 to C6 alcohol stream comprises at least one supply conduit for supplying a carbon dioxide-containing stream, with the conversion stage for converting the C1 to C6 alcohol stream being configured so as to convert the carbon dioxide deriving from the carbon dioxide-containing stream into carbon monoxide jointly with the conversion of the C1 to C6 alcohol stream.

In one variant, the plant comprises at least one recycle conduit for recycling to the conversion stage, at least a portion of the water from the mixture separated in the water separation stage.

The terms ‘comprising’ and ‘comprises’ as used herein are synonymous with ‘including’, ‘includes’ or ‘contains’, ‘containing’, and are inclusive or open-ended and are not intended to exclude additional characteristic features, elements or steps of methods that are not specified.

The terms ‘% by mass’ and ‘mass %’ have an equivalent meaning and refer to the proportion of the mass of a product relative to 100 g of a composition containing the same.

The boiling points as referred to herein are measured at atmospheric pressure, unless otherwise stated. An initial boiling point (hereinafter ‘IBP’) is defined as the temperature value from which a first vapor bubble is formed. A final boiling point (hereinafter ‘FBP’) is the highest temperature attainable during distillation. At this temperature, no more vapor can be transported to a condenser. The determination of the initial and final points is based on techniques known to the person skilled in the art, and several methods that are adapted as a function of the range of distillation temperatures are applicable, for example NF EN 15199-1 (2020 version) or ASTM D2887 for measuring the boiling points of petroleum fractions by gas chromatography; ASTM D7169 for heavy hydrocarbons; ASTM D7500, D86 or D1160 for distillates.

By default, the term ‘Cn to Cm’ stream, flow, fraction, etc, refers to a stream, flow, fraction, etc, that has a major quantity (for example greater than 50 mole %) of compounds having between n carbon atoms and m carbon atoms.

The term ‘Cn+’ stream, flow, fraction, etc, refers to a stream, flow, fraction, etc, that has a major quantity (for example greater than 50 mole %) of compounds having n carbon atoms or more than n carbon atoms.

The term ‘Cn−’ stream, flow, fraction, etc, refers to a stream, flow, fraction, etc, that has a major quantity (for example greater than 50 mole %) of compounds having n carbon atoms or less than n carbon atoms.

Unless otherwise indicated, the percentages used are mass percentages, and the pressures are absolute pressures.

### Obtaining the C1 to C6 Alcohol Stream

The C1 to C6 alcohol stream predominantly contains alcohols such as methanol, ethanol, propanols (n-propanol, i-propanol), butanols (n-butanol, i-butanol), pentanols (n-pentanols, i-pentanol) and hexanols.

It may comprise minor amounts of C6+ alcohols, and/or oxygenated compounds such as methyl ethyl ether; dimethyl ether; diethyl ether; diisopropyl ether; formaldehyde; dimethyl carbonate; dimethyl ketone; acetic acid; furans, tetrahydrofurans and mixtures thereof.

The C1 to C6 alcohol stream that forms the feedstock for the process advantageously comprises more than 80% by mass of C1 to C6 alcohols, preferably more than 90% by mass of C1 to C6 alcohols. It advantageously comprises more than 50% by mass of methanol, in particular more than 80% by mass of methanol.

In one variant, in the event that the conversion is carried out by means of a plurality of reaction zones having a fixed catalyst bed, a C2 to C6 alcohol stream is also advantageously added between two conversion reaction zones of conversion step (a), as will be described below.

The ratio of the mass flow rate of the C2 to C6 alcohol stream added between two reaction zones to the mass flow rate of the C1 to C6 alcohol stream entering the first reaction zone is, for example, less than 0.5, in particular between 0.05 and 0.5.

Preferably, the C1 to C6 alcohol stream and advantageously the additional C2 to C6 alcohol stream are obtained from a renewable source such as biomass (including the constituents and derivatives thereof) or from carbon monoxide or carbon dioxide, possibly captured, and from hydrogen advantageously produced from renewable energy sources such as solar energy, wind, geothermal energy, waves or currents and/or from energy whose production does not generate carbon dioxide such as nuclear energy.

The production pathways for producing the alcohols intended to form the C1 to C6 alcohol stream include, but are not limited to, for example:
anaerobic fermentation of sugars derived from biomass, in particular in order to obtain ethanol;
catalytic reaction of hydrogen with carbon dioxide or carbon monoxide, in order to obtain in particular methanol, ethanol and other alcohols;
catalytic reaction of hydrogen with carbohydrates in order to obtain in particular C1 to C6 alcohols;
Acetone-Butanol-Ethanol (ABE) fermentation, in order to obtain ethanol and n-butanol;
anaerobic fermentation of sugars derived from biomass, in order to obtain in particular propanol (iso or n), butanol (iso or n,) or isoamyl alcohol;
anaerobic fermentation of a mixture containing at least carbon monoxide, carbon dioxide and hydrogen, in order to obtain in particular ethanol, propanol (iso or n), butanol (iso or n) or isoamyl alcohol.

In one embodiment, in order to obtain alcohols, in particular ethanol derived from renewable sources by fermentation, the alcohol, in particular ethanol derived from renewable sources, may be obtained by ethanol fermentation in a bioreactor containing a culture of one or more microorganisms.

The ethanol derived from renewable sources may then advantageously be obtained by:
anaerobic fermentation of a substrate rich in biomass-derived sugars, or
anaerobic fermentation of a gas containing CO, which may or may not be derived from biomass.

In the case of anaerobic fermentation, ethanol may thus be produced by anaerobic fermentation of a substrate rich in biomass-derived sugars.

The sugars are composed of chains of 6 or 5 carbon atoms, such as glucose, sucrose (a dimer of glucose and fructose), xylose and arabinose.

This substrate may, for example, comprise or be sourced directly from plants for the agri-food industry, sugar cane, sugar beet, sugar sorghum; or obtained by depolymerization of the starch of maize, wheat, barley, rye, sorghum, triticale, potatoes, sweet potatoes, manioc, and/or the cellulose and hemicellulose of lignocellulosic biomass.

The sugar-rich substrate may also be derived from lignocellulosic biomass by a process comprising (i) a separation step of separating the lignin, cellulose and hemicellulose contained in the lignocellulosic biomass; followed by (ii) a conversion step of converting the cellulose and/or hemicellulose into sugars.

The production of this type of substrate from lignocellulosic biomass is well known to the person skilled in the art. Lignocellulosic biomass is essentially constituted of cellulose, hemicellulose and lignin. This biomass is obtained from agricultural and forestry residues or by-products of wood or crop processing, whether involving ligneous or herbaceous plants. This lignocellulosic biomass may also include distillers' grains and may be used to produce ethanol as described in the document EP2675778.

The first step (i) is a pre-treatment step which is used to de-compose the lignocellulosic matrix and free the cellulose and hemicellulose from the complex formed with the lignin by means of one or more pre-treatment processes. Among the existing pre-treatment processes, steam pre-treatment (or steam explosion), liquid hot water pre-treatment (hydrothermal pre-treatment), ammonia fiber explosion (AFEX), acid pre-treatment or alkaline pre-treatment are well-known. Steam explosion treatment consists of treating biomass, preferably shredded or ground beforehand, with high-pressure saturated steam at temperatures of around 160 to 240° C., and at pressures of 0.7 to 4.8 MPa. The effectiveness of the steam treatment may be enhanced by the addition of H₂SO₄, CO₂ or SO₂ as catalyst. In AFEX pre-treatment, the biomass is brought into contact with an anhydrous liquid ammonia feedstock in a ratio of 1:1 to 2:1 (1 to 2 kg ammonia/kg dry biomass) for a period of 10 to 60 minutes at 60-90° C. and pressures above 3 MPa. Hydrothermal pre-treatment is similar to steam explosion, but instead of steam it uses water in a liquid state at high temperatures. In the case of acid pretreatment, typically in the presence of dilute acid, an aqueous suspension of the cellulosic substrate is heated to the desired temperature and pretreated with preheated sulfuric acid (concentration levels <4% by mass) in a stainless steel reactor, the treatment process is carried out at a temperature of 140 to 215° C. The residence time varies from a few seconds to a few minutes depending on the treatment temperature. Lime pre-treatment is an inexpensive physico-chemical alkaline treatment that effectively enhances the digestibility of cellulosic biomass. Using 0.1 g of Ca(OH)2/g of biomass, the treatment process may be carried out over a wide temperature range from 25 to 130° C. The Organosolv process, which is a process for the delignification and/or saccharification of cellulosic materials and plant cultures, may also be used. In general, the Organosolv process involves the use of a mixture of water and a solvent such as alcohols or ketones, and sometimes other solvents of a non-polar nature, as well as an acidic compound in order to facilitate hydrolysis. A process of this type is described for example in the document U.S. Pat. No. 4,470,851A.

Step (ii) is a conversion step for converting cellulose and/or hemicellulose into sugars. It is also well known to those skilled in the art. It typically involves hydrolysis which may be catalyzed by using acid or by enzymes such as cellulases (produced, for example, by the strain Trichoderma reesei), xylanases, xylosidases and arabinofuranosidases.

The sugar-rich substrate is subsequently subjected to fermentation.

By way of example, this fermentation may be effected using micro-organisms, and in particular specialized yeasts that serve to optimize the yield of the production process, such as in particular the following yeasts: Ethanol Red® (Fermentie), Thermosacc® (Lallemand), Angel Super Alcohol® (Angel®), and Fali® (AB Mauric); the yeast strains Saccharomyces cerevisiae described in document FR3015985; the yeast strains Candida shehatae or Pichia stipitis; or any other suitable microorganism.

For anaerobic fermentation, ethanol may be produced by anaerobic fermentation of a gas comprising CO. The substrate is then a gaseous substrate (a gas) containing CO. This gaseous substrate may be a by-product of an industrial process.

In certain embodiments, the industrial process is selected from the group constituted of the: production of ferrous metal products, in particular steelworks; production of non-ferrous products; oil refining processes; gasification of coal and/or biomass or biochar; production of electrical energy; production of carbon black; production of ammonia; production of methanol; production of coke; catalytic cracking (in particular during catalyst regeneration carbon monoxide is produced); and reforming of methane. In these embodiments, the gaseous substrate can be captured from the industrial process before it is emitted into the atmosphere, using any appropriate method. Depending on the composition of the gas thus captured, it may also be desirable to treat it so as to remove any undesirable impurities, such as dust particles, before introducing it into the fermentation process. For example, the gas may be filtered or purified by known methods.

In other embodiments, the gaseous substrate may be derived from the gasification of biomass. The gasification process involves partial combustion of the biomass in a restricted supply of air or oxygen. The resulting gas typically comprises mainly CO and H2, with minimal volumes of CO2, methane, ethylene and ethane. For example, the biomass by-products obtained during the extraction and processing of food products, such as sugar from sugar cane or starch from corn or cereals, or non-food biomass waste generated by the forestry industry, may be gasified in order to produce a CO-containing gas that can be used in the present invention.

The gaseous substrate used typically has a significant proportion of CO. The CO content of the gaseous substrate is typically from 15% to 100% by volume, from 15% to 95% by volume, from 40% to 95% by volume, from 40% to 60% by volume, and from 45% to 55% by volume, or is within any range defined by two of these limits. Advantageously, the CO-containing gas may comprise 25%, 30%, 35%, 40%, 45%, 50%, 55% or 60% by volume of CO. Gases having lower CO content levels, such as 6% by volume, may also be suitable, particularly when H2 and CO2 are also present.

If the gaseous substrate contains CO, the gaseous substrate need not contain hydrogen, but this is not considered detrimental to the production of ethanol. The gaseous substrate may also contain CO2, for example, in a proportion of from 1% to 80% by volume, or from 1% to 30% by volume, or from 5% to 10% by volume, or within any range defined by any two of these limits.

Typically, the carbon monoxide is added to the fermentation reaction in a gaseous state, or in a liquid state. For example, carbon monoxide may be supplied in a liquid by saturation. For example, a liquid may be saturated with a carbon monoxide-containing gas and then this liquid may be added to a bioreactor. This can be performed using any standard methodology. By way of example, a microbubble dispersion generator (Hensirisak et. al. Scale-up of microbubble dispersion generator for aerobic fermentation; Applied Biochemistry and Biotechnolo RV Volume 101, Number 3/October, 2002) could be used.

In addition, it is often desirable to increase the CO concentration of the gas (or the partial pressure of CO in the gas) and thus boost the efficacy of the fermentation reactions using CO as a substrate. Increasing the partial pressure of CO in the gas increases the mass transfer of CO in a fermentation medium. The composition of the gas streams used to feed a fermentation reaction can have a significant impact on the efficacy and/or the costs of said reaction. For example, O2 can reduce the efficacy of an anaerobic fermentation process. Treating unwanted or unnecessary gases in the steps of a fermentation process before or after fermentation can increase the load in those steps (for example, when the gas stream is compressed before entering a bioreactor, unnecessary energy may be used to compress gases that are not necessary for the fermentation). Consequently, it may be desirable to treat the substrate streams, particularly substrate streams derived from industrial sources, in order to remove undesirable components and increase the concentration of desirable components.

Any microorganism capable of fermenting a gaseous substrate comprising CO in order to produce ethanol may be used in the present invention. By way of example, microorganisms of the genus Moorella, Clostridia, Ruminococcus, Acetobacterium, Eubacterium, Butyribacterium, Oxobacter, Methanosarcina, and Desulfotomaculum may be used.

By way of example, it is possible to use one or more micro-organism(s) of the genus Clostridium, including strains of Clostridium ljungdahlii, Clostridium carboxydivorans, Clostridium ragsdalei and Clostridium autoethanogenum; of the genus Moorella, including Moorella sp HUC22-1; of the genus Carboxydothermus; Moorella thermoacetica, Moorella thermoautotrophica; Ruminococcus productus; Acetobacterium woodii; Eubacterium limosum; Butyribacterium methylotrophicum; Oxobacter pfennigii; Methanosarcina barkeri; Methanosarcina acetivorans; or Desulfotomaculum kuznetsovii. Other specific examples of microorganisms are anaerobic carboxydotrophic bacteria. Examples of usable strains are described in the document WO201226833.

It should be noted that the invention may be applied to a mixed culture of two or more microorganisms.

With regard to the fermentation medium and conditions, regardless of the nature of the substrate (gaseous or otherwise) used, in order for the fermentation of ethanol to occur by means of the growth of one or more microorganisms, an appropriate nutrient medium must be introduced into the bioreactor in addition to a substrate, under appropriate conditions. A nutrient medium will contain appropriate components, such as vitamins and minerals, that are sufficient to enable the growth of the micro-organism used. The reaction conditions to be considered are temperature, flow rate of the medium, pH, redox potential of the medium, agitation rate (if using a continuous agitation reactor), inoculum level, maximum substrate concentrations and rates of introduction of substrate into the bioreactor in order to ensure that the substrate level does not become limiting, as well as maximum product concentrations in order to avoid product inhibition. The optimum reaction conditions will depend in part on the particular micro-organism used. The processes for culturing micro-organisms are known in the art and the person skilled in the art knows how to optimize the culture conditions for each micro-organism, based on its nature. Examples of fermentation conditions that are appropriate for anaerobic fermentation of a substrate comprising CO are detailed in WO2007/117157, WO2008/115080, WO2009/022925 and WO02/08438.

The fermentation reactions may be carried out in any suitable bioreactor. In certain embodiments of the invention, the bioreactor may comprise a first growth reactor in which the microorganisms are cultured, and a second fermentation reactor, in which broth obtained from the growth reactor is introduced and in which most of the fermentation product (for example, ethanol) is produced.

The fermentation will result in a fermentation broth comprising a desirable product (ethanol) and/or one or more by-products (such as acetate and butyrate when the substrate is a CO-containing gas) as well as microorganism cells, in a nutrient medium.

The recovery of ethanol may include the continuous withdrawal of a portion of the broth and the recovery of ethanol from the removed portion of the broth.

For example, the removed portion of the ethanol-containing broth may be passed through a separation unit in order to enable separation, for example by filtration, of the bacterial cells from the broth and production of a cell-free ethanol-containing permeate, and return of the micro-organism cells to the bioreactor.

In certain embodiments, the recovery of ethanol and/or of one or more other product(s) or by-product(s) produced in the fermentation reaction comprises the continuous removal of a portion of the broth and the separate recovery of ethanol and of one or more other product(s) from the removed portion of the broth.

As an example, ethanol may be recovered from the fermentation broth using methods such as filtration, distillation or fractional evaporation, pervaporation, and extractive fermentation. Distillation of ethanol from a fermentation broth provides an azeotropic mixture of ethanol and water (i.e. 95% ethanol and 5% water). Anhydrous ethanol can then be obtained by using molecular sieve ethanol dehydration technology, which is also well known in the art.

Ethanol derived from renewable sources may also be obtained from biomass by conversion of a CO/H2-rich synthesis gas, said synthesis gas being derived from biomass.

For example, the biomass may be gasified in order to produce a CO/H2-rich synthesis gas (or also known as ‘syngas’), with this syngas then being converted into methanol in the presence of a catalyst. A process of this type is described, for example, in the document WO2012003901.

The biomass used to produce syngas may in particular include wood fuels sourced from forests and natural woodlands (for example, sawdust); agricultural residues (for example, rice husks, straw manure); energy crops that are grown exclusively for energy production (for example, maize and oil palm); urban waste (for example, wood waste, rice, straw manure); municipal waste (for example, municipal solid waste and wastewater); and biomass fuel derived from waste (for example, wood pellets).

It is also possible to obtain methanol derived from renewable sources. The term ‘methanol derived from renewable sources’ is used to refer to methanol obtained from biomass.

The biomass may in particular include wood fuels sourced from forests and natural woodlands (for example, sawdust); agricultural residues (for example, rice husks, straw manure); energy crops that are grown exclusively for energy production (for example, maize and oil palm); urban waste (for example, wood waste, rice, straw manure); municipal waste (for example, municipal solid waste and wastewater); and biomass fuel derived from waste (for example, wood pellets).

Methanol derived from renewable sources may in particular be obtained by converting a CO/H2-rich synthesis gas, said synthesis gas being derived from biomass.

For example, the biomass may be gasified in order to produce a CO/H2-rich synthesis gas (or also known as ‘syngas’), with this syngas then being converted into methanol in the presence of a catalyst. A process of this type is described, for example, in the document WO2018134853A1.

A synthesis gas that is suitable for subsequent conversion into methanol may also be obtained by partial oxidation in the presence of oxygen of a biogas containing methane and CO2, this biogas resulting, for example, from the anaerobic digestion of biomass in the presence of one or more microorganism(s). A process of this type is, for example, described in the document WO2019060988A1.

The alcohols intended to form the C1 to C6 alcohol stream and optionally the additional C2 to C6 alcohol stream can also be obtained from carbon dioxide, in particular captured carbon dioxide.

There are a number of existing conversion pathways. One example that may be cited is the catalytic conversion of carbon dioxide into methanol in the presence of hydrogen.

Another pathway consists of converting carbon dioxide into carbon monoxide by electroconversion or by reacting the gas with reverse water in the presence of hydrogen.

The carbon monoxide is then converted by catalytic conversion into methanol, in the presence of hydrogen.

The hydrogen used for the various operations described above is obtained in particular by steam methane reforming, by reaction of the gas with water, or is produced by electrolysis from renewable energy sources such as solar energy, wind, geothermal energy, waves or currents.

### Step (a) Conversion of the C1 to C6 Alcohol Stream

The conversion of the C1 to C6 alcohol stream comprises, for example, dehydration, carbon-carbon coupling and/or aromatization of at least one C1 to C6 alcohol.

The dehydration of the C1 to C6 alcohols, the carbon-carbon coupling and/or and the aromatization thereof may be performed simultaneously.

For dehydration of methanol, the latter is generally converted into dimethyl ether, which is dehydrated in order to produce olefins having at least two carbon atoms. In the case of methanol and/or dimethyl ether, the carbon-carbon coupling takes place during dehydration.

The C2-C6 alcohols may be dehydrated in order to produce olefins containing the same number of atoms. Dehydration reactions for dehydrating alcohols in order to produce alkenes have been known for a long time (J. Catal. 7, p. 163, 1967 and J. Am. Chem. Soc. 83, p. 2847, 1961). A number of available solid acid catalysts can be used for the dehydration of alcohols ((Stud. Surf. Sci. Catal. 51, p. 260, 1989), EP0150832, Bulletin of the Chemical Society of Japan, vol 47(2), 424-429 (1974)). However, γ-aluminas are the most commonly used, particularly for longer chain alcohols (with three or more carbon atoms). Indeed, catalysts with a higher acidity, such as silica-aluminas, zeolites, heteropoly-acids or resin catalysts, can promote the displacement of double bonds, backbone isomerization and other olefin interconversion reactions.

In addition, aromatization of C1 to C6 alcohols occurs via oligomerization of olefin intermediates, cyclization of a chain having at least 6 carbon atoms and dehydrogenation of cycloparaffins into the corresponding aromatics.

The mechanism of reactions in the conversion of C1 to C6 alcohols involves acid catalysis. The catalyst can provide a proton to activate the molecules, alcohols and/or olefins via protonated intermediates. The stability of protonated intermediates or alkylcarbenium ions depends on the inductive effect of the substituents. The tertiary carbenium ions are the most stable, while the primary carbenium ions are the least stable. Thus, tertiary carbenium ions are the most readily formed, while reactions involving the formation of primary carbenium ions are slow. The primary carbenium ions tend to transform into secondary or tertiary carbenium ions.

The addition of carbenium ions to alkenes is the key step in the oligomerization of alkenes, and the addition of carbenium ions to aromatic hydrocarbons is the basis for the alkylation of aromatics with alkenes.

Either the hydride transfer provides a pathway for converting a neutral molecule into a carbenium ion, or the successive hydride transfer from the alkene to the carbenium ion results in the formation of aromatic compounds. The following reactions demonstrate this mechanism for propylene:

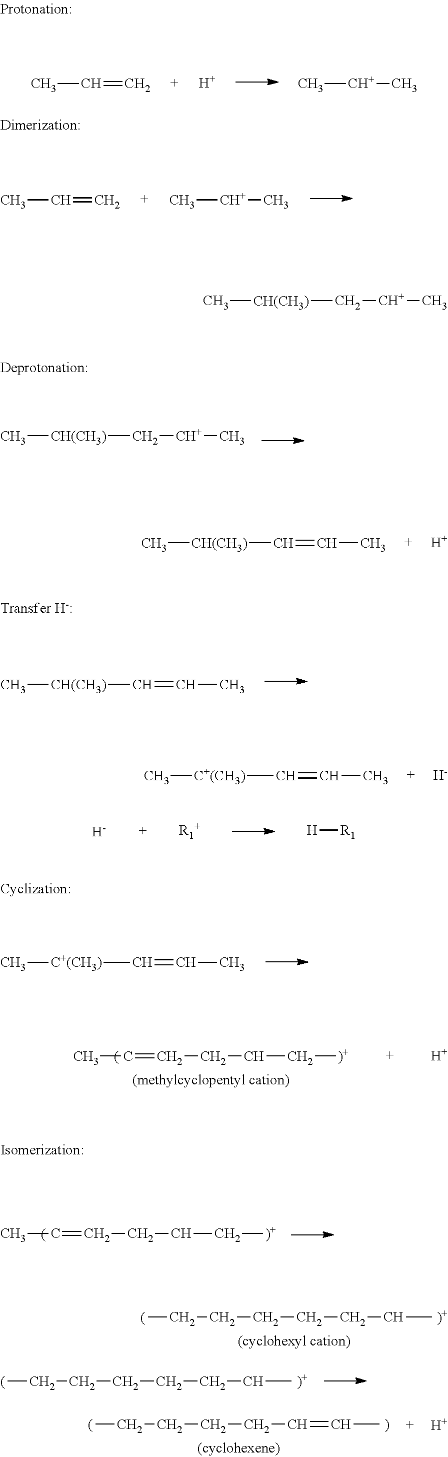

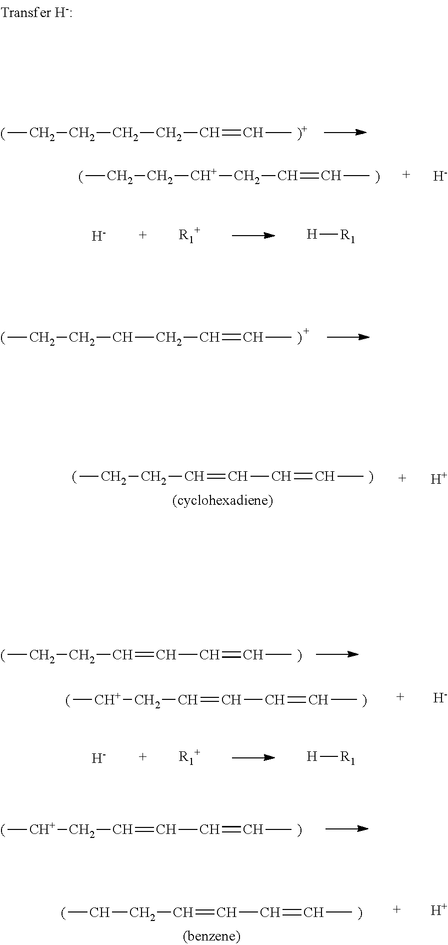

Overall, in order to form an aromatic ring, three dihydrogen molecules (six atoms) need to be removed, and these three hydrogen molecules will, according to the hydride transfer mechanism, form three paraffins from the olefins.

The hydride transfer mechanism is typically brought about on catalysts having only one acid function, and often requires severe conditions that are also conducive to the formation of coke.

The production of aromatics from olefins (produced from alcohols) takes place on acid catalysts essentially via hydride transfer from one olefin to another olefin. This produces more unsaturated molecules (and ultimately aromatics) and paraffins. These paraffins are not transformable, as they are too inert to be converted under the optimal operating conditions for the conversion of C1-C6 alcohols. It is possible to add a dehydrogenating catalytic function, which enables aromatization by producing molecular hydrogen.

If the catalyst also has a dehydrogenating function, the reactive intermediates can be converted into the corresponding aromatics by dehydrogenation. This is particularly the case with bifunctional catalysts, that have an acid function and a dehydrogenating function. The dehydrogenating function may be provided by metals from groups VIB, VIIIB, IB, and IB and mixtures thereof; preferably gallium, zinc or mixtures thereof.

In general, the dehydrogenation reactions for dehydrogenating olefins are thermodynamically limited and require high temperatures. In order to promote the dehydrogenation pathway, a further reagent may be added to conversion step (a) in order to shift the thermodynamic equilibrium.

In a surprising manner, according to the invention, this reagent is carbon dioxide, which can be converted into carbon monoxide and water:

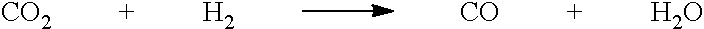

The CO2, CO and H2 molecules can be separated from the other hydrocarbons and recycled to the synthesis of alcohols, by fermentation of syngas and by catalytic conversion of syngas in order to make methanol therefrom.

The process then involves, in conversion step (a) for converting the C1 to C6 alcohol stream, adding a carbon dioxide-containing stream, and then concurrently converting the carbon dioxide to carbon monoxide in conversion step (a) for converting the C1 to C6 alcohol stream.

Advantageously, the carbon dioxide-containing stream comprises more than 5% by mass of carbon dioxide, in particular more than 10% by mass of carbon dioxide.

In the feedstock supplied to conversion step (a), the mass ratio of carbon dioxide supplied in the carbon dioxide stream to the C1 to C6 alcohols supplied in the C1 to C6 alcohol stream is between 5% and 75%.

The carbon dioxide-containing stream is added, for example, in admixture with the C1 to C6 alcohol stream; or where a plurality of reaction zones are arranged in series to implement the conversion step, either between two reaction zones or within a given reaction zone.

Preferably, more than 2 mol %, in particular more than 6 mol %, of the carbon dioxide is converted into carbon monoxide in conjunction with the conversion of the C1 to C6 alcohol stream in conversion step (a).

The main product of the acid-catalyzed dehydration of ethanol and/or methanol is ethylene and/or propylene and water.

More generally, a mixture containing paraffins, olefins, aromatics, and water is produced.

The paraffins include n-paraffins, i-paraffins, and cycloparaffins.

According to the invention, in the mixture of paraffins, olefins, aromatics and water produced in conversion step (a), the ratio of the mass of C3+ olefins to the total mass of olefins is greater than or equal to 0.80, in particular greater than 0.82, preferably greater than or equal to 0.85, the ratio being calculated on the basis of the dry stream, after separation of the water.

Advantageously, not taking into account the water circulating in a water recycle process, the mixture produced containing paraffins, olefins, aromatics, and water contains more than 10% by mass of water, in particular between 10% and 60% by mass of water depending on the alcohol composition in the mixture of C1 to C6 alcohols. In the event that only methanol is present, the water content of the product mixture (excluding recycle) is between 55% by mass and 60% by mass.

Advantageously, on a dry basis, excluding water and any recycled material, the mixture produced containing paraffins, olefins, aromatics, and water contains more than 2% by mass of aromatics, in particular more than 6% by mass of aromatics, in particular between 6% and 30% by mass of aromatics.

On a dry basis, excluding water, it advantageously contains:
less than 5% by mass of methane, in particular between 0.1% by mass and 4% by mass of methane;
less than 5% by mass of dimethyl ether, in particular less than 1% by mass of dimethylether;
less than 5% by mass of residual C1 to C6 alcohols, in particular less than 1% by mass of residual C1 to C6 alcohols;
less than 15% by mass of ethylene, in particular between 5% by mass and 10% by mass of ethylene;
more than 30% by mass of propylene, in particular between 35% by mass and 60% by mass of propylene;
less than 15% by mass of paraffins, in particular between 3% and 8% by mass of paraffins;
more than 10% by mass of C4 to C7 olefins, in particular between 25% and 40% of C4 to C7 olefins;
more than 6% of aromatics, in particular between 6% and 10% of aromatics, in particular less than 3% of C9+ aromatics.

The production of light olefins (ethylene and propylene) from a mixed alcohol feedstock in an oxygenates-to-olefins process is described, for example, in U.S. Pat. No. 7,288,689. The said patent proposes various processes for producing C1 to C4 alcohols, optionally in a mixed alcohol stream, and optionally for converting the alcohols into light olefins.

Conversion by dehydration and aromatization makes it possible to obtain, from C1-C6 alcohols using a composite catalyst, light olefins having at least 2 carbon atoms as well as aromatics, the process comprising the following steps:
a) providing a catalyst comprising molecular sieves containing at least 10 oxygen atom sized pores (10-MR) or larger in their microporous structure;
b) in the event of the conversion being carried out in a reaction zone comprising a fluidized bed, providing a reaction zone and a catalyst regeneration zone; said catalyst circulating in both zones, such that at least a portion of the regenerated catalyst passes through the reaction zone and at least a portion of the catalyst in the reaction zone passes through the regeneration zone;
or/and in the event of the conversion being carried out in a reaction zone with at least one fixed bed, a catalyst regeneration step for regenerating the catalyst in situ, either by directing the stream to be reacted to a previously regenerated reaction zone, or by stopping the conversion in the reaction zone;
c) contacting the C1 to C6 alcohols in the reactor with the catalyst under conditions that are effective for converting at least a portion of the feedstock so as to form a reactor effluent comprising a mixture of paraffins, olefins, aromatics, and water.

The catalyst may be a mixture of two or more catalysts and optionally a binder.

It is desirable to have a conversion of substantially 100% of the alcoholic compound in the reactor. This conversion rate is adjusted by optimizing the contact time, reaction temperature and catalyst regeneration frequency.

In one specific embodiment, the weight hourly space velocity (hereinafter WHSV) of the alcohol in the reaction zone is about 0.5 h⁻¹ to about 10 h⁻¹, advantageously from about 1 h⁻¹ to about 6 h⁻¹.

The molecular sieves included in the composition of the catalyst are selected from the list of molecular sieves with crystalline structure MFI, MOR, MEL, clinoptilolite, FER, FAU, MWW, BETA, MCM-41, ZSM-21, ZSM-22, ZSM-23, ZSM-42, ZSM-57, LTL or a mixture thereof. Preferably, the molecular sieve selected is a zeolite, a crystalline aluminosilicate selected from the group including MFI, MOR, MEL, clinoptilolite, FER or a mixture thereof. More preferentially, in the case of MFI, the molecular sieve is preferably a ZSM-5 zeolite. In one further embodiment, the molecular sieve is preferably obtained without the addition of a structuring agent. Further examples are described by the International Zeolite Association (Atlas of Zeolite Structure Types, 1987, Butterworths).

Crystalline silicates (also called zeolites) are microporous crystalline inorganic polymers based on a framework of XO4 tetrahedra linked to each other by sharing of oxygen ions, where X may be trivalent (e.g. Al, B, . . . ) or tetravalent (e.g. Ge, Si, . . . ). The crystal structure of a crystalline silicate as determined by X-Ray diffraction is defined by the specific order in which a network of tetrahedral units are linked together. The size of the crystalline silicate pore openings is determined by the number of tetrahedral units, or, alternatively, oxygen atoms, required for forming the pores and the nature of the cations that are present in the pores. They possess a unique combination of the following properties: high internal surface area; uniform pores with one or more discrete sizes; ion exchangeability; good thermal stability; and ability to adsorb organic compounds. Since the pores of these crystalline silicates are similar in size to many organic molecules of practical interest, they control the ingress and egress of reactants and products, resulting in particular selectivity in catalytic reactions. Crystalline silicates with the MFI structure possess a bidirectional intersecting pore system with the following pore diameters: a straight channel along [010]:0.53-0.56 nm and a sinusoidal channel along [100]:0.51-0.55 nm. Crystalline aluminosilicates with the MEL structure possess a bidirectional intersecting straight pore system with straight channels along [100] having pore diameters of 0.53-0.54 nm.

The molecular sieves used in the present invention (in H+ or NH4+ form) have an initial Si/Al ratio that is advantageously between 4 and 500, preferentially from 4 to 100, or more preferentially from 4 to 30. Conversion to the H+ or NH4+ form is known per se and described in U.S. Pat. Nos. 3,911,041 and 5,573,990. The Si/Al atomic ratio is measured by chemical analysis, for example by NMR. It includes only those Al that form part of the framework structure of the molecular sieve.

According to a first embodiment, said zeolite is a phosphorus-modified zeolite produced by a process comprising in the order indicated:
Selection of a molecular sieve as described in the list above;
Introduction of P under conditions that are effective to advantageously introduce at least 0.05% by mass of P by adding an aqueous solution containing a phosphorus precursor;
Possible separation of the solid from the aqueous liquid as applicable;
Optional washing or optional drying, or optional drying followed by washing;
Calcination.

Optionally, the process for producing said phosphorus-modified zeolite includes the steps of steam heat treatment and leaching. The method consists of steaming/heat treatment followed by leaching.

It is generally known to the person skilled in the art that steam treatment of zeolites, results in aluminum that leaves the zeolite framework and resides as aluminum oxides within and outside the pores of the zeolite. This transformation is known as dealumination of zeolites and this term will be used throughout the text.

In the steam treatment step, the temperature is preferably from 400° C. to 870° C., more preferentially from 480° C. to 760° C. The pressure is preferably atmospheric and the partial pressure of water may range from 13 kPa to 100 kPa. The steam atmosphere preferably contains from 5% to 100% by volume of steam with from 0% to 95% by volume of an inert gas, preferably nitrogen. The steam treatment is preferably carried out for a period of 0.01 hours to 200 hours, advantageously from 0.05 hours to 200 hours, more preferentially from 0.05 hours to 50 hours. The steam treatment tends to reduce the amount of tetrahedral aluminum in the crystalline silicate framework by forming alumina.

The treatment of the steam-treated zeolite with an acid solution results in dissolution of the extra-framework aluminum oxides. This transformation is known as leaching and this term will be used throughout the text. The leaching can be made with an organic acid such as citric acid, formic acid, oxalic acid, tartaric acid, malonic acid, succinic acid, glutaric acid, adipic acid, maleic acid, phthalic acid, isophthalic acid, fumaric acid, nitrilotriacetic acid, hydroxyethylenediaminetriacetic acid, ethylenediaminetetracetic acid, trichloroacetic acid trifluoroacetic acid or a salt of such an acid (e.g. the sodium salt) or a mixture of two or more of such acids or salts. The other inorganic acids may comprise an inorganic acid such as nitric acid, hydrochloric acid, methansulfuric acid, phosphoric acid, phosphonic acid, sulfuric acid or a salt of such an acid (e.g. the sodium or ammonium salts) or a mixture of two or more of such acids or salts. Leaching with an acidic aqueous solution containing a phosphorus source is advantageously carried out under reflux conditions, i.e. at the boiling temperature of the solution. The quantity of said acid solution is advantageously between 2 liters and 10 liters per kg of molecular sieve. A typical leaching period is around 0.5 hours to 24 hours. Advantageously, the acidic aqueous solution containing the P source in the leaching step has a pH of 3, advantageously 2, or lower. Advantageously, said aqueous acid solution is a solution of phosphorus acids, a mixture of phosphorus acids and organic or inorganic acids or mixtures of salts of phosphorus acids and organic or inorganic acids. The phosphorus acids or corresponding salts may be phosphate ([PO4]³⁻, being basic), phosphite ([HPO3]²⁻, being dibasic) or hypophosphite ([H2PO2]−, being monobasic). Unexpectedly, a greater amount of phosphorus remains in the solid molecular sieve material than would be expected based on the typical volume of pores in the molecular sieve and assuming that the molecular sieve pores are filled with the phosphorous acid solution used. The two factors, namely dealumination and P retention, stabilize the framework aluminum in the zeolite framework, thus avoiding further dealumination. This leads to higher hydrothermal stability, the tuning of molecular sieve properties and the adjustment of acidic properties, thereby increasing molecular sieve selectivity.

Then the zeolite is separated, advantageously by filtration, and optionally washed. A drying step may be envisaged between the filtering and washing steps. The solution after the washing can be either separated, by way of example, by filtering from the solid, or evaporated. P can be introduced by any means or, by way of example, according to the recipe described in U.S. Pat. Nos. 3,911,041, 5,573,990 and 6,797,851. The separation of the liquid from the solid is advantageously done by filtration at a temperature between 0° C.-90° C., centrifugation at a temperature between 0° C.-90° C., evaporation or equivalent. Optionally, the zeolite may be dried after separation before washing. Advantageously the said drying is carried out at a temperature between 40° C. and 60° C., advantageously for a period of 1 h-10 h. This drying may be carried out either under static conditions or in a gas stream. Air, nitrogen, or any inert gas may be used. The washing step may be carried out either during filtration (separation step) with a portion of cold water (<40° C.) or hot water (>40 but <90° C.), or the solid may be subjected to an aqueous solution and treated under reflux conditions for a period of 0.5 h to 10 h followed by evaporation or filtration. The final calcination step is advantageously carried out at a temperature of 400° C.-700° C. either under static conditions or in a gas stream. Air, nitrogen, or any inert gas may be used.

According to one embodiment of the invention, the phosphorus-modified zeolite is produced by a process comprising in the order indicated:
Selection of a molecular sieve as described in the list above;
Steam heat treatment at a temperature ranging from 400° C. to 870° C. for a period of 0.01-200 hours;
Leaching with an aqueous acid solution under conditions that are effective for removing a substantial portion of Al from the zeolite;
Introduction of P with an aqueous solution containing the P source under conditions that are effective for advantageously introducing at least 0.05% by mass of P
Separation of the solid from the liquid;
An optional washing step or an optional drying step or an optional drying step followed by a washing step;
A calcination step.

Optionally between the steam heat treatment step and the leaching step there is an intermediate step such as, by way of example, contact with silica powder and drying.

Advantageously the final P-content is at least 0.05 mass % and preferably between 0.3 and 7 mass %. Advantageously at least 10% of Al, relative to parent zeolite MFI, MEL, FER, MOR and clinoptilolite, have been extracted and removed from the zeolite by the leaching. Then, the zeolite is either separated from the washing solution or is dried without separation from the washing solution. The said separation is advantageously achieved by filtration. Then the zeolite is calcined, by way of example, at 400° C. for a period of 2 to 10 hours.

The residual P content is adjusted by the P concentration in the aqueous acid solution containing the P source, the drying conditions and a washing procedure if any. A drying step may be envisaged between the filtering and washing steps.

The catalyst consisting of a phosphorus-modified zeolite can be the phosphorus-modified zeolite itself or it can be the phosphorus-modified zeolite formulated into a catalyst by combining with other materials that provide additional hardness or catalytic activity to the finished catalyst product.

According to a second embodiment, the catalyst of the process is a composite catalyst produced by a process comprising the following steps:
a) Selection of a molecular sieve from the list as previously defined above
b) Contacting the molecular sieve with a metal silicate comprising at least one alkaline earth metal, such that the composite contains at least 0.1 mass % of silicate.

The molecular sieve is preferably brought into contact with the metal silicate by one of the following two methods:
During the catalyst formulation step, by mechanical mixing of the molecular sieve with the metal silicate forming a precursor to be used in the formulation step;
Physical mixing of the previously formulated molecular sieve and the metal silicate previously formulated in situ in the reaction medium intended for use in performing the conversion.

Said molecular sieve and/or said composite catalyst containing the molecular sieve and the metal silicate can be post-treated by calcinations, reductions, or hydrothermal steam treatment. When using zeolites as molecular sieve components, the phosphorus may be introduced before, simultaneously with, or after mixing with the metal silicate.

In one particular embodiment of the invention, the molecular sieve may be modified either before or after the introduction of the metal silicate. Preferably, the molecular sieve has undergone a certain form of modification prior to the introduction of the metal silicate. The term “modification” is used herein to indicate that the molecular sieve may have undergone steam heat treatment, leaching (e.g. acid leaching), washing, drying, calcination, impregnation or some form of ion exchange. This means that at least a portion of the cations initially included in the crystal structure may be replaced by a wide variety of other cations using techniques that are well known in the art. The replacement cations may be hydrogen, ammonium or other metal cations, including mixtures of such cations.

The selected molecular sieve is then formulated into a composite catalyst so as to comprise at least 10 mass % of a molecular sieve as described herein and at least one metal silicate comprising at least one alkaline earth metal, such that the composite comprises at least 0.1 mass % of silicate.

At least one of the metal silicates included in the composite catalyst comprises at least one alkaline earth metal, preferably Ca. The metal silicates are insoluble in water, and the alkaline earth metal ions, in particular calcium, are polyvalent and have a large radius in the hydrated state. Thus, without intending to be bound by theory, it is thought that the ion exchange reaction with the molecular sieve occurs very slowly, as the alkaline-earth metal ion must lose many of its strongly coordinated water molecules in order to penetrate the micropores of the sieve. As a result, the alkaline-earth ions expose only the acidic sites situated on the external surface of the molecular sieve, thus increasing the selectivity of the catalyst.

Furthermore, without intending to be bound by theory, it is believed that the presence of silicate anions further enhances the catalytic properties of the composite catalyst. For example, the silicate anions can provide silicon atoms to repair defects in the molecular sieve. This can thus lead to additional stabilization of the catalyst under severe hydrothermal conditions.

As a result, the metal silicate acts as a catalyst promoter. The metal silicate may comprise more than one alkaline earth metal selected from Ca, Mg, Sr and Ba.

The metal silicates may also comprise other metals selected from one or more of the following: Ga, Al, Ce, In, Cs, Sc, Sn, Li, Zn, Co, Mo, Mn, Ni, Fe, Cu, Cr, Ti and V. Preferably, the other metal is selected from one or more of the following: Al, Mg, Ce, Co and Zn or mixtures thereof. These bi-, tri- or polymetallic silicates can be synthesized by any method known in the art. For example this may be by ion exchange in solution or in the solid state (Labhsetwar et al., Reactivity of Solids, vol. 7, issue 3, 1989, 225-233).

The silicate anion may be present in any form in the solid metal silicate. Examples include SiO₃²⁻, SiO4⁴⁻, Si₂O₇⁶⁻, Si₃O₁₀⁸⁻ and the like.

The preferred catalyst promoter is a calcium silicate with a very open and accessible pore structure. An even more preferred catalyst promoter comprises a synthetic crystalline calcium silicate hydrate having a chemical composition of Ca6Si6O17(OH)2 which corresponds to the known mineral xonotlite (having a molecular formula 6CaO·6SiO2·H2O).

Generally, a synthetic calcium silicate hydrate is synthesized by a hydrothermal process under autogenous pressure. A particularly preferred synthetic calcium silicate hydrate is commercially available from the company Promat, Ratingen, Germany, under the brand name Promaxon.

Other examples of metal silicates comprising alkaline earth metals include CaAl2Si2O8, Ca2Al2SiO7, CaMg(Si2O6)x and mixtures thereof.

Prior to mixing with the molecular sieve, said metal silicate compounds may be modified by calcination, steam treatment, ion exchange, impregnation or phosphatization. Said metal silicates may be in the form of individual compounds or be part of mixed compounds.

The metal silicate may be contacted with the molecular sieve by a formulation step for simultaneously formulating a mixture of the metal silicate with the molecular sieve or an in situ mixture of separately formulated materials in the reaction medium prior to effecting the conversion.

The said contact may be effected by mechanical mixing of the molecular sieve with the metal silicate comprising an alkaline-earth metal. This can be carried out by any known mixing process. The mixing may be performed over a period of time ranging from 1 minute to 24 hours, preferably from 1 minute to 10 hours.

If not carried out in the in situ conversion reactor, it may be carried out in a batch mixer or in a continuous process, such as in an extruder, for example a single or twin screw extruder at a temperature of 20° C. to 300° C. under vacuum or high pressure. The said contact may be effected in an aqueous or non-aqueous medium. Prior to the formulation step, other compounds that facilitate formulation may be added, such as thickening agents or polyelectrolytes that enhance the cohesion, dispersion and flow properties of the precursor. In the case of oil-drop drying or spray drying, a rather liquid fluid (high water content) is prepared. In one further embodiment, the contacting is effected in the presence of phosphorus-containing compounds. In one particular embodiment, the contacting is effected in an aqueous medium at a pH of less than 5, more preferentially less than 3.

According to a third embodiment, the catalyst of the process is a molecular sieve modified with phosphorus (P) and with an alkaline-earth metal or rare-earth metal (M) (MP modified molecular sieve) which is produced by a process comprising the following steps:
a) selecting at least one molecular sieve chosen from
   a P-modified molecular sieve that contains at least 0.3% by mass of P
   a molecular sieve modified with P before or during step b) introducing at least 0.3% by mass of P
b) contacting said molecular sieve with a compound containing an alkaline-earth metal or rare-earth metal (M-containing compound) so as to introduce at least 0.05% by mass of the alkaline-earth metal or rare-earth metal M.

Optionally, the contacting of the molecular sieve with the P-containing compound and the M-containing compound can be effected simultaneously.

The introduction of the alkaline-earth metal or rare-earth metal (M) is effected by contacting the molecular sieve with a solution of one or more M-containing compounds. Said solution may contain a higher concentration of alkaline-earth metal or rare-earth metal than that found in the final MP modified molecular sieve.

The molecular sieve is selected from the list previously described above.

Prior to P-modification and/or alkaline earth metal- or rare earth metal-modification (M-modification), the molecular sieve may undergo other treatments, including steam heat treatment, leaching (for example, acid leaching), washing, drying, calcination, impregnation or ion exchange. In addition or as a variant, these steps may also be carried out during or after P-modification. The term “ion exchange steps” herein is understood to indicate that at least some of the cations initially included in the crystalline structure are replaced by a wide variety of other cations in accordance with techniques well known in the art. The replacement cations may be hydrogen, ammonium or other metal cations, including mixtures of such cations.

The modification of molecular sieves with phosphorus is known per se. This modification is effected by treating molecular sieves with P compounds in aqueous or non-aqueous media, by chemical deposition in vapor phase of organic P compounds, or by impregnation. The catalyst may be pre-formulated with or without a binder. Preferred P compounds typically used for this purpose may be selected from the group comprising phosphoric acid, NH4H2PO4 or (NH4)2HPO4. The M-containing compound may be selected from among organic compounds, salts, hydroxides and oxides. These compounds may also contain phosphorus. It is essential for these compounds to be present in solubilized form, prior to being contacted with the molecular sieve, or by forming a solution in contact with the molecular sieve.

The final M/P molar ratio in the MP molecular sieve is preferably less than 1.

According to one particular embodiment of the invention, the molecular sieve may be modified with phosphorus by the process comprising the following steps, in the order indicated:
steam heat treatment of the molecular sieve at a temperature ranging from 400° C. to 870° C. for a period of 0.01 h-200 h;
leaching with an acidic aqueous solution containing the P source under conditions that are effective for removing a substantial portion of Al from the molecular sieve and for introducing at least 0.3% phosphorus by mass of the molecular sieve; an additional modification may then be effected according to the following steps, in the order indicated:
separation of the solid from the liquid;
an optional washing step or an optional drying step or an optional drying step followed by a washing step;
a calcination step.

Preferably, the separation, the optional washing and drying steps, and the calcination are carried out after introduction of the M-containing compound into the molecular sieve. The metal M may be any alkaline earth metal or rare earth metal. Preferably, the alkaline earth metal is Ca. However, it is also possible to use Mg, Sr and Ba. Possible rare earth metals include La and Ce.

Advantageously, the final P content of the molecular sieve is at least 0.3 mass % and preferably between 0.3 mass % and 7 mass %. Advantageously, at least 10 mass % of Al has been extracted and removed from the molecular sieve by leaching. The residual P content is adjusted by the P concentration in the leaching solution, separating the conditions during the separation of the solid from the liquid and/or the optional washing procedure during which impregnation and/or adsorption can also take place. A drying step may be envisaged between the separation and/or washing steps.

The molecular sieve is then either separated from the washing solution, or dried without separation from the washing solution. The said separation is advantageously effected by filtration. The molecular sieve is then calcined, for example, at 400° C. for a period of 2 hours to 10 hours.

The M modification of the molecular sieve is carried out either on a molecular sieve that has already been P modified, or during/after the P modification process. The P modification may be effected as described above, wherein the sieve is dealuminated by steam heat treatment, and subsequently leached with a P-containing acid solution. In this case, advantageously, treatment of the molecular sieve with the M-containing solution is carried out after the leaching or washing step, that is to say, after the phosphorous compound has been added and the P modification has taken place, and before the separation step.

However, the introduction of M into the molecular sieve may also be envisaged:
during the leaching step;
before the washing step but after leaching and drying;
on calcined molecular sieves contacted with P;
on a molecular sieve that has not been leached in order to introduce P but has been contacted with P during the washing step.

The introduction of M on molecular sieves may be effected either by impregnation, or by adsorption from an aqueous solution of M containing compounds.

The M-containing compound can be introduced at temperatures ranging from ambient temperature to the boiling point of the solution. The concentration of the M-containing compound in the solution is at least 0.05 M, preferably between 0.05 and 1.0 M. The amount of alkaline earth metal or rare earth metal (M) in the MP molecular sieves can vary from at least 0.05 mass %, preferably from 0.05 mass % to 7 mass %, better still from 0.1 mass % to 4 mass %.

Prior to the formulation of the composite catalyst, the molecular sieve may undergo further treatment processes including steps such as steam treatment, leaching (e.g. acid leaching), washing, drying, calcination, impregnation, and ion exchange. In addition or as a variant, these steps may also be carried out after formulation of the catalytic composite.

The alkaline earth metal or rare earth metal M is preferably selected from one or more of: Mg, Ca, Sr, Ba, La, Ce. More preferentially, M is an alkaline earth metal. More preferably, M is Ca. Particularly in the case of P modification by vaporization and leaching, M can be a rare-earth metal such as La and Ce.

The M-containing compound is preferably in the form of an organic compound, a salt, a hydroxide or an oxide. The compound is preferably in solubilized form when brought into contact with the molecular sieve. Alternatively, the solution of the M-containing compound may be formed after the molecular sieve is contacted with said compound.

Possible M-containing compounds include compounds of metal M such as sulfate, formate, nitrate, M-metal acetate, halides, oxyhalides, borates, carbonate, hydroxide, oxide, and mixtures thereof. These may be for example, calcium sulfate, formate, nitrate, acetate, halides, oxyhalides, borates, carbonate, hydroxide, oxide and mixtures thereof.

The M-containing compound may also include other metals selected from one or more of: Mg, Sr, Ba, Ga, Al, Ce, In, Cs, Sc, Sn, Li, Zn, Co, Mo, Mn, Ni, Fe, Cu, Cr, Ti, and V. The M-containing compounds may additionally also include phosphorus.

These M-containing compounds, which are poorly soluble in water, can be dissolved so as to form a well-solubilized solution by heating and/or modifying the pH of the solution by adding phosphoric-, acetic- or nitric acid or the corresponding ammonium acid; the salts of said acids. The concentration of the M-containing compound is at least 0.05 M.

Alkaline-earth metals and rare-earth metals M, in particular Ca, have a large hydration sphere radius in the hydrated state. Thus, without intending to be bound by theory, it is believed that the ion exchange reaction with the acidic sites located within the microporous structures of the molecular sieve occurs very slowly. As a result, the M metal selected exposes only the acid sites located on the external surface of the molecular sieve, thereby increasing the selectivity of the catalyst.

In the case of P-modified molecular sieves, the M modification leads to the formation of mixed M-Al-phosphates on the external surface. Given that the phosphorus is more strongly bound to the alkaline earth or rare earth metal M than to Al, this modification leads to stabilization of the phosphorus on the external surface of the molecular sieve where the phosphorus is most labile. However, it is essential that all M atoms on the external surface be saturated with phosphorus. This can be ensured in the presence of excess phosphorus and by the presence of M in solution form, which serves, for example, to wash out excess phosphorus and prevent clogging of the micropore inlet.

Before, after or simultaneously with the formulation step to form the composite, other components may optionally be mixed with the molecular sieve which may or may not be MP-modified. In one particular embodiment, the MP-modified or unmodified molecular sieve may be combined with other materials that confer additional hardness or catalytic activity to the finished catalytic product. The materials, which can be mixed with the molecular sieve, may be various inert or catalytically active matrix materials and/or various binder materials. Such materials include clays, quartz, alumina or alumina sol, silica or silica sol and/or metal oxides such as titanium oxide, zirconia, and mixtures thereof. In one embodiment, certain binder materials may also be used as diluents in order to control the conversion rate of the product feed and thus enhance selectivity. According to one embodiment, the binders also improve catalyst attrition under industrial operating conditions. Natural clays that may be used as binders are, for example, clays from the kaolin family or the montmorillonite family. Such clays can be used in their raw state as mined, or they may be subjected to various treatments prior to use, such as calcination, acid treatment or chemical modification. In addition to the above, other materials that may be incorporated into the composite catalyst of the invention include various forms of metals including rare earth or alkaline earth metals, phosphates (for example metal phosphates, where the metal is selected from one or more of: Ca, Ga, Al, Ca, Ce, In, Cs, Sr, Mg, Ba, Sc, Sn, Li, Zn, Co, Mo, Mn, Ni, Fe, Cu, Cr, Ti and V). Examples of possible phosphates include amorphous calcium phosphate, monocalcium phosphate, dicalcium phosphate, dehydrated dicalcium phosphate, a- or p-tricalcium phosphate, octacalcium phosphate, hydroxyapatite, etc; zirconia, silica-thorine, silica-beryllium, silica-titanium, calcium-alumina. Examples of ternary binder compositions include for instance calcium-silica-alumina or silica-alumina-zirconia. These components are effective in increasing catalyst density and augmenting the strength of the catalyst formulation. The catalyst that can be used in fluidized-bed reactors has a substantially spherical shaped form, generally formed by atomization.

Generally, in the event of using a fluidized bed as a reactor, the size of the catalyst particles may vary from about 20 μm to 500 μm, more preferably from 30 μm to 100 μm. The size of the molecular sieve crystals contained in the composite catalyst, is preferably less than about 10 μm, more preferably less than about 5 μm, and all together most preferably less than about 2 μm. Generally speaking, in the event of using a fixed bed, the size of the catalyst particles may vary from around 0.5 to 5 mm in the form of beads, extrudates (length between 1 and 10 mm), either cylindrical or trilobed or quadrulobed in shape. The amount of molecular sieve contained in the final catalyst composite ranges from 10% by mass to 90% by mass of the total catalyst composite, preferably from 20% by mass to 70% by mass.

According to one other embodiment, unmodified molecular sieves were first formulated with a binder and matrix materials and then modified with phosphorus silicates and alkaline earth metal silicates. According to another particular embodiment, the molecular sieves were optionally dealuminated and then modified with phosphorus during the formulation step. The alkaline earth metal silicate may be introduced during the formulation step or on the formulated solid.

According to one preferred embodiment, the molecular sieves have first optionally been dealuminized and modified with phosphorus, and then formulated. The introduction of the metal is done simultaneously with the phosphorus modification step and/or on the already formulated catalyst.

After formulation, the composite catalyst can undergo further treatments including further steps of steam treatment, leaching, washing, drying, calcination, impregnation and ion exchange. If the molecular sieve has not been modified with phosphorus prior to the formulation step of formulating the mixture, i.e. the step of introducing the metal silicate into the molecular sieve, this may be done following the said step. According to a particular feature of this embodiment the molecular sieve is a phosphorus-modified (P-modified) zeolite. Said phosphorus-modified (modified with P) zeolite has already been described above.

According to one other embodiment, the unmodified molecular sieve has first been formulated with a binder and matrix material and then subsequently modified with phosphorus and metals. According to one particular embodiment, the molecular sieves have optionally been desaluminated and thereafter modified with phosphorus during the formulation step. The introduction of the metal may be done during the formulation step or on the formulated solid. According to one preferred embodiment, the molecular sieve optionally has first been desalinated and modified with phosphorus, and thereafter formulated. The introduction of the metal may be done simultaneously with the step of phosphorus modification and/or on the formulated catalyst.

The final catalyst containing a phosphorus-modified zeolite advantageously has a 27Al NMR signature, between 35 ppm and 45 ppm, which is characteristic of the presence of an ALPO structure. The mass content of the said AlPO4 structure in the catalyst may be up to 99% by mass and is advantageously between 10% and 98% by mass.

The presence of this ALPO structure is characterized by means of the following method, illustrated in FIG. 9. The measurement is carried out by Magic-angle spinning (MAS) in solid-state nuclear magnetic resonance (NMR) spectroscopy performed on the Bruker Avance 500 spectrometer, with a 4 mm zirconia MAS probe at a rotational speed of 15 kHz. In order to obtain quantitative MAS spectra, a single-pulse excitation was applied using a short pulse length of 0.6 psec. Each spectrum results from 5000 scans separated by a delay of 0.5 seconds. Chemical shifts of 27Al spectra were referenced to AlCl3 solution (0.1M, (0 ppm)).

In the case where there is only one zeolitic aluminum source in the catalyst, the content of the AlPO4 phase is estimated directly by a ratio of the signal area at 35 ppm-45 ppm (centered at 39 ppm in FIG. 9) in the 27Al MAS to a total spectrum area between −50 ppm and 100 ppm.

In the case where the binder contains aluminum and phosphorus, the content of the AlPO4 phase in the zeolite is estimated by a ratio of the signal area at 35 ppm-45 ppm in the 27Al MAS to the total spectrum area between −50 ppm and 100 ppm after subtraction of the binder signal intensities.

In order to enhance selectivity towards the formation of aromatics, the catalysts as described above may be further modified by the addition of one or more metals selected from those in Group IB (e.g. Zn), IIIB (e.g. Ga; transition metals in Group VIIIB (e.g. Fe and/or Ni and/or Pt), Group VIB (e.g. Mo), Group IB (e.g. Cu and/or Ag; or even from the lanthanide group (e.g. La). The introduction of the metal(s) may be done using various methods known to the person skilled in the art, such as, but not limited to, ion exchange, dry or equilibrium impregnation, grafting, chemical vapor deposition (CVD). The introduction of the metal(s) is effected by means of one or more solutions containing the metals in the form of salts. The metal salts are first dissolved in advance in the treatment solution; the metal counterions are selected from among sulfates, nitrates, carbonates, hydroxides, phosphates, carboxylates (e.g. formate, acetate, propionate) and dicarboxylates (e.g. oxalate, malonate, succinate).

After the introduction of the metal(s), various different treatments may be applied, including drying, calcination, steam heat treatment.

The metal content is generally between 0% by mass and 5% by mass, preferentially between 0% by mass and 2.5% by mass. The selected metal typically may be Ga3+ or Zn2+, in the presence/absence of other metals such as Pt.

The above-mentioned catalyst may also be modified by the addition of B, in an amount of from 0.1% by mass to 5% by mass, preferentially from 0.1% by mass to 1% by mass, more preferentially from 0.1% by mass to 0.5% by mass, in the presence or absence of the other above-mentioned metals.

In one other embodiment, the conversion process for converting oxygenates is carried out over a catalyst comprising a zeolite with pore sizing of 10 oxygen atoms (10-MR) or more, modified by the adding of B either before, after, or simultaneously with the formulation step of formulating the final catalyst. Preferentially, the catalyst for example may be a B-modified ZSM-5.

The B content in the final catalyst is between 0.1% by mass and 5% by mass, preferentially between 0.1% by mass and 1% by mass, more preferentially between 0.1% by mass and 0.5% by mass.

Advantageously, the zeolite contained in the final catalyst has a Si/Al atomic ratio—as measured by chemical analysis (for example by NMR), taking into account only those Al that form part of the framework structure of the molecular sieve—of between 4 and 500, preferentially between 5 and 200, or more preferentially between 12 and 150.

With regard to the conversion stage in which conversion step (a) is implemented, the C1-C6 alcohol stream is contacted with the catalyst described above in a reaction zone of at least one reactor under operating conditions conducive to producing the mixture containing paraffins, olefins, aromatics, and water as defined above.

In this step (a), which converts alcohols, the mixture can generally be produced in a temperature range from 300° C. to 600° C., notably between 330° C. and 550° C., in particular between 350° C. and 500° C., or between 410° C. and 580° C.

The pressure may also vary over a wide range. Preferred pressures are within the range from about 100 kPa to about 5 MPa, the most preferred range being from about 150 kPa to about 1.0 MPa. The above pressures refer to the partial pressure of oxygen-containing organic compounds.

Conversion step (a) can be carried out in a single reaction zone or in a plurality of reaction zones arranged in series or parallel. After a certain operating time period, the catalyst is to be regenerated.

In particular, a plurality of reactors may be used in order to ensure that the exothermicity of the reaction is controlled in a manner so as to avoid excessive temperatures. Preferably, the maximum temperature difference within the same reactor should not exceed 100° C. and preferably 75° C.

With respect to the type of reactor(s), either isothermal or adiabatic fixed-bed, moving-bed, and/or fluidized-bed reactors may be used.

The conversion reaction may be carried out in continuous mode in a configuration comprising a succession of fixed beds connected in series, in at least one operating reactor wherein the raw feed material passes from one fixed bed to the other with cooling therebetween; and at least one analogous reactor connected in parallel, which undergoes a catalyst regeneration operation.

Advantageously, as described above, an additional C2-C6 alcohol stream is added between two fixed beds in order to control exothermicity.

The conversion reaction may also be carried out in continuous mode in a configuration comprising a series of mobile beds connected in series, wherein the raw feed material passes from one mobile bed to the other with cooling therebetween, and wherein the catalyst is mobile and circulates between the reactor and a catalyst regeneration zone.

The conversion reaction may also be carried out in continuous mode in a configuration comprising one fluidized bed which forms a reaction zone where the reaction takes place, and one fluidized bed which forms a regeneration zone where regeneration takes place (for example, by controlled combustion in the presence of oxygen); or fluidized beds connected in series, wherein the raw feed material passes from one bed to the other with cooling therebetween, and wherein the catalyst is mobile and circulates between the reactor and the catalyst regeneration zone.

Fluidized beds offer significant advantages when reactions are in particular highly exothermic. Once the bed solids have been fluidized, the solids inside the bed behave like a liquid. The size, shape, formation, ascent rate and coalescence of gas bubbles in fluidized beds are quantitatively similar to those of gas bubbles in liquids.

The liquid-like behavior of a fluidized bed thus provides the ability to handle solids like a fluid, thereby making it possible to supply and/or extract solids. Rigorous mixing in a fluidized bed also enables a uniform temperature to be achieved, even for highly exothermic reactions, and thus allows for more flexible control of the reactor. Rigorous mixing also ameliorates contact between solids and fluids, thus augmenting heat and mass transfer.

There are numerous variants of fluidized beds, which have been described in available technical manuals (e.g. Handbook of fluidization and fluid-particle system, Taylor&Francis Group LLC, 2003). The fluidization phenomena of gas-solid systems are highly dependent on the types of powders used. Several classifications exist, all based on Geldart's original work. Many catalysts used in fluidized bed systems are particles of Group A, which are characterized by a dense phase expansion after minimum fluidization and before the onset of bubbling. Gas bubbles appear at minimum bubbling velocity.

Fluidization regimes can be classified into two broad categories: fluidization in particulate mode (smooth), and in aggregative mode (bubbling). In particulate fluidization, the solid particles are generally dispersed in a relatively uniform manner in the fluidization medium, with no easily identifiable bubbles. Particulate fluidization is therefore sometimes also referred to as homogeneous fluidization. In heterogeneous or aggregative fluidization, voids (bubbles) containing no solids are generally formed and are observed in a bubbling fluidized bed or in a bed exhibiting “slugging”. For gas-solid systems, there exist a number of distinct fluidization regimes: fixed bed, particulate fluidization, bubbling fluidization, slugging fluidization, and turbulent fluidization; for each of these, the relevant criteria are available. Where the operating speed is higher than the transport speed, such that recycling of entrained particles is necessary in order to maintain a bed, additional fluidization regimes are possible.

$$Particulate regime : Umf \leq U < Umb$$

For Group A powders, the fixed bed expands homogeneously (particulate fluidization) above the minimum fluidization velocity (Umf) and no bubbles are observed as long as the velocity remains below the minimum bubbling velocity (Umb).

Bubbling regime: Umb≤U<Ums

Bubbles appear when gas velocity is increased above the minimum bubbling velocity (Umb). Gas bubbles form above the distributor, coalesce and grow. The bubbling regime is characterized by the coexistence of a bubble phase and a dense/emulsion phase. Most of the fluidizing gas is present in the form of bubbles and, consequently, the gas velocity through the dense phase is very low.

$$Slugging regime : Ums \leq U < Uc$$

Given the bed's high height-to-diameter ratios, the bed provides sufficient time for the bubbles to coalesce into larger ones. When the bubbles reach approximately the size of the bed's cross-section, the bed enters the “slugging” regime, with a periodic passage of large bubbles forming plugs and a large, regular fluctuation in pressure drop across the bed. The velocity Uc corresponds to the bed operating conditions where the plugs reach their maximum diameter and where the amplitude of the fluctuation in pressure is at its highest.

Transition to turbulent regime: Uc≤U<Uk

When the gas velocity is continuously increased beyond the said velocity Uc, the large bubbles begin to fragment into smaller bubbles with a smaller pressure fluctuation. This velocity is denoted as Uk, and characterizes the transition between the bubbling regime and the turbulent regime.

$$Turbulent regime : Uk \leq U < Utr$$

Up until the transport velocity (Utr), the bed is in turbulent regime. Bubbles or voids continue to be present, although they are less distinguishable in the dense suspension. In this regime, interactions between the gas voids and the dense/emulsion phase are vigorous, and ensure effective gas-solid contact.

$$Fast fluidization regime : U > Utr$$

Above the transport velocity (Utr), the particles begin to be entrained, and continuous operation is no longer possible without replacing or recycling the entrained and transported particles. Fast fluidized beds are typically characterized by a dense phase region at the bottom, close to the distributor, coexisting with a dilute phase region at the top. The velocity of the particles increases with elevation in the bed, and thus the density of the bed decreases.

$$Pneumatic transport : U >> Utr$$

All particles introduced at the bottom of the fluidized bed are transported in a dilute phase with a concentration that varies along the height of the bed.

A typical example of a reaction zone is the fluidized bed with riser used in fluid catalytic cracking (FCC) applications. Risers are vertical pipes with a high height-to-diameter ratio (>10), and the ideal riser approximates plug flow conditions, such that the catalyst and fluid phase pass through the riser with minimal back-mixing.

In a transport fluidized bed reactor (either fast fluidization or pneumatic transport), a core-annular flow can occur, wherein a dilute, high-velocity core is surrounded by a denser, slower ring. When the circulating mass flows are low, the solids in the ring flow downwards across the wall. When the circulating mass flows are high, the solids in the ring flow upwards along the wall. This non-uniform flow phenomenon leads to inefficient gas-solid contact and sub-optimal catalyst performance, and significant back-mixing of the gas and solids will occur, particularly when there is downward flow descending in the wall region. For fast fluidization, the interiors are used to redistribute the axial and radial gas-solid flow structure, that is to say, to enhance the uniformity of the gas-solid flow structure in space and thus promote radial gas-solid exchange. Fluidized transport reactors require the catalyst particles to be recirculated to the bottom of the reactor. This offers the possibility of controlling the density of the catalyst in the fluidized bed by recirculating more or less catalyst.

At the bottom of the fluidized bed, the feed fluid is distributed homogeneously over the cross-section of the reactor vessel. At the top of the reaction zone, the reaction vapors are separated from the entrained catalyst by means of baffles, a disengagement zone and cyclones. The catalyst is collected, stripped of any remaining hydrocarbons and advantageously returned to the bottom of the fluidized bed zone via vertical manifolds (“standpipe”) and valves.

For the exothermic reaction such as the conversion carried out in step (a), it is preferable to have a homogeneous temperature across the catalyst bed (radially and axially) in order to avoid hot spots and to properly control the catalytic reaction. This can be achieved by rapid recirculation and possibly re-mixing of the catalyst in the reactor vessel.

The means for controlling the average temperature of the reaction consists of introducing the feed into the reaction zone at a temperature below the average bed temperature and/or removing the heat from the catalyst bed via heat exchange. This heat exchange may be effected by means of: internal heat exchange tubes through which a cooling medium circulates and removes heat from the reactor vessel; or external heat exchange by circulating the hot catalyst, collected at the top of the reactor, around the heat exchanger tubes and recirculating the cooled catalyst within the reactor vessel.

With regard to catalyst regeneration, the conversion reactor for converting the C1 to C6 alcohols also includes a regeneration zone (or regenerator), the main objective of which is to eliminate coke deposits on the catalyst by combustion with oxygen. The regenerators are fast fluidized bed systems. Generally speaking, the regenerator comprises a dense catalyst bed at the bottom of the vessel and a more dilute bed near the top of the vessel.

There are two types of regenerators, which operate in either partial combustion mode or total combustion mode. In partial combustion mode, an amount of air less than the stoichiometric quantity is supplied to the regenerator. The carbon is for the most part converted into carbon monoxide, with only a portion being converted into carbon dioxide. Ideally, all the oxygen is consumed so that no oxygen is present in the flue gases. The CO/CO2 ratio in the flue gases is generally between 0.5 and 2.0. In total combustion mode, excess air is supplied to the regenerator. Ideally, all the carbon contained in the coke is converted into carbon dioxide, and no carbon monoxide is present in the flue gases. The residual oxygen content in the flue gases is between 1.0% by volume and 3.0% by volume on a dry basis.

Partial combustion regenerators offer several advantages over total combustion regenerators, particularly when the catalyst is sensitive to high temperatures and the steam environment: (i) it is possible to burn more coke for a given air flow rate, as the amount of air required is less than the stoichiometric amount, and (ii) a small amount of combustion heat is released, which enables moderate temperature control and better preservation of catalytic activity in the presence of the steam produced by the combustion of hydrogen.

A potential drawback of the partial-combustion regenerator is the higher amount of coke remaining on the regenerated catalyst. In the event of regeneration by total combustion, the amount of carbon remaining on the catalyst is low and the restoration of catalytic activity is higher. The potential drawback of total combustion regenerators is that the heat released is greater due to the total combustion reaction, thus resulting in a more irreversible loss of catalytic activity. The use of two-stage regeneration can reduce catalyst deactivation. In two-stage regeneration, the first stage operates at a moderate temperature in order to burn off mainly the hydrogen present in the coke, which has a higher reaction rate, as well as a portion of the carbon. In the second stage, using excess air, the remaining carbon is burnt at a higher temperature producing carbon dioxide, and thanks to the absence of water vapor in the second-stage regenerator, catalyst deactivation at high temperatures can be minimized.

The use of fluidized beds provides the ability to very precisely control the exothermicity of the reaction, while offering continuous catalyst regeneration that promotes productivity and simplifies operations.

One or more diluents may be present in the C1 to C6 alcohol stream feeding the reaction zone, for example, in an amount from 1 mol % to 95 mol %, based on the total number of moles of all feed and diluent components introduced into the reaction zone.

Typical diluents include, but are not limited to, helium, argon, nitrogen, hydrogen, water (optionally recycled), paraffins, alkanes (in particular methane, ethane and propane), aromatics, and mixtures thereof. Preferred diluents are water and nitrogen. The water may be injected in liquid or vapor form. The use of a diluent can offer two advantages. The first advantage is to reduce the partial pressure of the alcohol and thus enhance selectivity for light olefins, mainly propylene. Generally speaking, the lower the alcohol partial pressure, the higher the selectivity for light olefins, and conversely, the higher the partial pressure, the higher the selectivity for heavy olefins such as butenes and pentenes. With regard to the yield of light olefins, there exists an optimum yield as a function of partial pressure, reaction temperature, feed hourly space velocity and catalyst properties.

The second advantage of using a diluent is that it can act as a heat sink for the conversion of exothermic alcohols. Thus, the higher the specific molar heat capacity, the greater the amount of heat that can be absorbed by the diluents. This second advantage may be less significant in the case of fluidized-bed reactors, as the latter are known to be excellent reactors for operating at an almost homogeneous temperature throughout the catalyst bed. It is preferable for the diluents to be easily separated from the light olefin products, preferably by a simple phase separation. Consequently, water is a preferred diluent. The diluents may be added at a rate ranging from 1 mol % to 95 mol % of the combined feedstock (C1 to C6 alcohol stream+diluents), preferably from 10 mol % to 75 mol %.

### Step (b) Separation of Water from the Mixture

Water from the mixture containing paraffins, olefins, aromatics, and water produced in conversion step (a) is separated from the mixture in a water separation stage, in order to form a water-depleted mixture.

The water separation step is preceded by a cooling step for cooling the effluent from step (a), condensing the water as well as a portion of the hydrocarbons. The temperature at which this step is carried out is generally between 20° C. and 100° C.

The separation is based, for example, on the difference in density and solubility between the water and the rest of the hydrocarbons. It is generally carried out in a three-phase separator flask that serves to separate a water-rich aqueous phase (hereinafter referred to as the water separated from the mixture), a liquid hydrocarbon phase, and a gaseous hydrocarbon phase.

The water-depleted mixture contains less than 5% by mass, preferably less than 1% by mass, of the water present in the mixture produced in step (a).

The water separated from the mixture advantageously contains less than 10% by mass of hydrocarbons.

The water separated from the mixture is optionally at least partially recycled to conversion step (a).

In the event that conversion step (a) is carried out using a plurality of successive fixed catalyst beds, the water separated from the mixture is optionally recycled upstream of the fixed catalyst beds or between two fixed catalyst beds.

In this case, the mass ratio of recycled water to the C1 to C6 alcohol stream in the feedstock supplied to conversion step (a) is advantageously between 0 and 3, preferably between 0.05 and 2.

In the event that conversion step (a) is carried out using at least one fluidized bed, the recycled water is optionally re-injected into the fluidized bed.

In this case, the mass ratio of recycled water to the C1 to C6 alcohol stream in the feedstock supplied to conversion step (a) is advantageously between 0 and 1, preferably between 0.05 and 0.5.

In both of the preceding cases, the recycled water forms a stream which controls the exothermicity of the reaction, reduces hydrocarbon partial pressures, and modifies the acidity of the catalyst, which in turn enhances olefin selectivity.

The water that has been separated but not recycled to conversion step (a) is advantageously treated by stripping in a stripping column in order to separate the hydrocarbons contained therein into an extracted hydrocarbon stream. The extracted hydrocarbon stream is re-injected into separation step (b).

In the event that conversion step (a) is carried out using at least one fluidized bed, a C4− hydrocarbon stream may optionally be added to the fluidized bed to supplement or replace the recycled water. This stream is formed, for example, by at least a portion of a C1-C2 hydrocarbon fraction separated from the water-depleted mixture, as described below. This stream also controls the exothermicity of the reaction.

Advantageously, the C1 to C6 alcohol stream is introduced in conversion step (a) at a temperature at least 5° C. higher than the bubble point of the C1 to C6 alcohol stream, and preferably lower than the temperature of the conversion reaction implemented in step (a), for example at least 50° C. lower than the temperature of the conversion reaction, advantageously at least 100° C. lower than the temperature of the conversion reaction.

The heating of this stream absorbs the calories released by the conversion of the alcohols.

### Optional Steps for the Separation of C1-C2 Hydrocarbons and C3 Hydrocarbons

Advantageously, the water-depleted mixture is introduced into a separation stage comprising at least one distillation column (hereinafter referred to as a deethanizer) for separating the C1-C2 hydrocarbons (methane, ethane, ethylene) and other gaseous molecules that are lighter than the C2s, such as CO, CO2 and hydrogen, from the remainder of the water-depleted mixture.

The distillation column operates, for example, at a pressure greater than 20 barg, and preferably greater than 30 barg.

A C1-C2 hydrocarbon fraction is extracted from the top of the column. It contains more than 50% by mass of C1-C2 hydrocarbons and other gaseous molecules such as CO, CO2 and hydrogen.

The C1-C2 hydrocarbon fraction preferably contains more than 90% by mass, in particular more than 95% by mass, of the C1-C2 hydrocarbons and other gaseous molecules such as CO, CO2 and hydrogen contained in the water-depleted mixture.

The C1-C2 hydrocarbon fraction is at least partly conveyed to an ethylene recovery unit, for example in a steam cracker. Thus, ethylene, even if produced in a minor quantity during conversion step (a), can be recovered.

Advantageously, following the conversion of the carbon dioxide added, in the conversion step (a), into the carbon dioxide-containing stream, the carbon dioxide, carbon monoxide, and hydrogen present in the C1-C2 hydrocarbon fraction are optionally separated from the other hydrocarbons, in particular by cryogenic distillation, membrane separation or pressure swing adsorption, and combinations thereof.

These compounds are then advantageously recycled to a preliminary alcohol synthesis step, notably by fermentation of syngas and by catalytic conversion of the syngas in particular to produce methanol.

In particular, the methanol or ethanol produced in this way form a portion of the C1 to C6 alcohol stream which is subsequently converted in the conversion step (a).

In one advantageous variant, particularly applicable in the case of a conversion step (a) carried out using at least one fluidized bed, a portion of the C1 to C2 hydrocarbon fraction is recycled to conversion step (a) for converting the C1 to C6 alcohol stream in the form of a recycle stream. For example, the ratio of the mass flow rate of the portion of the C1-C2 hydrocarbon fraction recycled to conversion step (a), to the mass flow rate of the C1-C2 hydrocarbon fraction drawn from the distillation column is less than 1 and is notably between 0.1 and 0.8.

The C3+ hydrocarbon fraction is recovered at the bottom of the column. It comprises more than 90% by mass of the C3+ hydrocarbons contained in the water-depleted mixture.

On a dry basis, it advantageously contains:
less than 5% by mass of C1-C2 hydrocarbons;
less than 15% by mass of paraffins, in particular between 3% and 10% by mass of paraffins;
more than 40% by mass of C3 to C7 olefins, in particular between 50% and 80% of C3 to C7 olefins,
less than 5% by mass of C8+ olefins, in particular between 0.1% by mass and 4.0% by mass of C8+ olefins; and/or
more than 6% of aromatics, in particular between 6% and 20% of aromatics.

The C3 to C7 olefins generally contain propylene. In particular, the C3+ hydrocarbon fraction contains in some cases more than 30% by mass of propylene.

In one embodiment, the C3+ hydrocarbon fraction is sent directly to the oligomerization and alkylation step. By way of a variant, in one particular embodiment, an additional separation step of separating C3− hydrocarbons, in particular propylene, is carried out in a second distillation column. The said separation enables the recovery of propylene.

The second distillation column, for example, operates at a pressure greater than 5 barg, and preferably greater than 10 barg. It produces at the top of the column, a C3-hydrocarbon fraction, containing more than 50% by mass of propylene, and at the bottom, a C4+ hydrocarbon fraction.

The C3− hydrocarbon fraction preferably contains more than 90% by mass, in particular more than 95% by mass of the C3− hydrocarbons contained in the C3+ hydrocarbon fraction from the first distillation column.

The C4+ hydrocarbon fraction comprises more than 90% by mass of the C4+ hydrocarbons contained in the water-depleted mixture.

On a dry basis, it advantageously contains:
less than 5% by mass of C3− hydrocarbons;
less than 25% by mass of paraffins, in particular between 10% and 15% by mass of paraffins;
more than 15% by mass of C4 to C7 olefins, in particular between 25% and 40% of C4 to C7 olefins;
less than 2% by mass of C8+ olefins, in particular between 0.5% by mass and 1.0% by mass of C8+ olefins; or
more than 6% of aromatics, in particular between 7% and 20% of aromatics, in particular less than 5% of C9+ aromatics.

### Step (c) Oligomerization of Olefins from the Water-Depleted Mixture; and Step (d) Alkylation of Aromatics from the Water-Depleted Mixture

In the first embodiment, oligomerization step (c) for oligomerizing olefins from the water-depleted mixture and alkylation step (d) for alkylating aromatics from the water-depleted mixture are carried out jointly together in the same one or more reactor(s) of a same given reaction stage.

Advantageously, the hydrocarbon feed load formed by the C3+ hydrocarbon fraction or the C4+ hydrocarbon fraction described above is oligomerized for its olefins and alkylated for its aromatics by means of contacting with an acid catalyst.

For example, a multi-reactor plant may be used in which the exothermicity of the reaction can be controlled in a manner such as to avoid excessive temperatures. Preferably, the maximum difference in temperatures within a same given reactor should not exceed 100° C. and preferably 75° C.

The one or more reactor(s) may be of such types as isothermal- or adiabatic fixed or moving bed reactors. The olefin oligomerization reaction and the aromatics alkylation reaction may be carried out in continuous mode in a configuration comprising a series of fixed beds connected in series, in at least one reactor in operation wherein the raw feed material passes from one bed to the other with cooling therebetween; and at least one analogous reactor connected in parallel, which undergoes a catalyst regeneration operation. The olefin oligomerization reaction and the aromatics alkylation reaction may be carried out in continuous mode in a configuration comprising a series of mobile beds connected in series, wherein the raw feed material passes from one bed to the other with cooling therebetween and wherein the catalyst is mobile and circulates between the reactor and the catalyst regeneration zone.

Preferably, the above-noted steps are carried out jointly by means of at least two successive reactors. In the case of fixed-bed reactors, alternatively a single reactor may contain a plurality of catalytic beds with cooling systems between the beds, or else be provided with a quench flow injection in order to lower the temperature between the beds.

The reaction conditions of the first reactor are selected so as to enable the conversion of a portion of olefinic compounds with low carbon numbers (C3-C8) into intermediate olefins (C8+), and the alkylation of aromatics by light olefins.

Advantageously, the first reactor comprises a first catalytic zone and operates at high temperatures, for example greater than or equal to 200° C., and preferably less than 350° C.; and a pressure of between 25 bar and 60 bar.

The second reactor preferably operates at temperatures and pressures selected so as to promote the conversion of a portion of the low-carbon (C3-C8) olefinic compounds into intermediate olefins (C8+), and the alkylation of aromatics by light olefins. The effluent from the first reactor, comprising unreacted olefins, intermediate olefins, aromatics, water and possibly other compounds such as paraffins, and possibly a reducing gas, then undergoes oligomerization and/or alkylation in this second reactor comprising a second catalytic zone, which makes it possible to obtain a distillate-rich, heavier hydrocarbon effluent.

A cooling section is advantageously provided between two successive reactors, and optionally a flash drum.

The mass flow rate through the oligomerization reactor(s) is advantageously sufficient so as to enable a relatively high conversion, without being too low in order to avoid undesirable parallel reactions.

The weight hourly space velocity (WHSV) of the feed is, for example, from 0.1 h⁻¹ to 20 h⁻¹, preferably from 0.5 h⁻¹ to 10 h⁻¹, even more preferably from 0.8 h⁻¹ to 5 h⁻¹.

The temperature at the inlet of the one or more reactor(s) is advantageously sufficient so as to enable a relatively high conversion, without being excessively high in order to avoid undesirable parallel reactions.

The temperature at the inlet of the or each reactor is, for example, from 150° C. to 400° C., preferably from 180° C. to 350° C., even more preferably from 200° C. to 290° C.

The pressure through the one or more reactor(s) for olefin oligomerization and aromatics alkylation is advantageously sufficient so as to enable a relatively high conversion, without being too low in order to avoid undesirable parallel reactions.

The pressure through the or each reactor for example, ranges from 8 bara to 100 bara, preferably from 10 bara to 85 bara, and more preferably from 25 bara to 75 bara (bars, absolute pressure).

As regards the nature of the catalyst, a first family of catalysts used comprises an acid catalyst of a type such as either amorphous or crystalline aluminosilicate, or a silicoaluminophosphate, in H+ form, selected from the following list and whether or not containing alkaline or rare-earth elements:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, BEA (zeolite Beta), MOR (mordenite), FAU (faujasite type zeolite), LTL (zeolite L), zeolite Omega and the family of microporous materials composed of silica, aluminum, oxygen and optionally boron.

Prior to its use, the zeolite may be subjected to a variety of treatments, which may include: ion exchange; modification with metals; steaming; acid treatments or any other appropriate de-alumination method; silicon surface passivation by silicon deposition; or any combination of the aforementioned treatments.

The content of alkalis or rare earths ranges from 0.05% by mass to 10% by mass, preferentially from 0.2% by mass to 5% by mass. Preferentially, the metals utilized are Mg, Ca, Ba, Sr, La, Ce, used individually or in mixtures thereof.

A second family of catalysts used includes phosphorus-modified zeolites optionally containing an alkali or a rare earth. In this case, the zeolite may be selected from the following list:

MFI (ZSM-5, silicalite-1, boralite C, TS-1), MEL (ZSM-11, silicalite-2, boralite D, TS-2, SSZ-46), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, FAU, LTL, BEA (zeolite Beta), MOR.

Prior to its use, the zeolite may be subjected to a variety of treatments, which may include: ion exchange; modification with metals; steaming; acid treatments or any other appropriate de-alumination method; mesoporization treatments; silicon surface passivation by silicon deposition; or any combination of the aforementioned treatments.

The content of alkalis or rare earths ranges from 0.05% by mass to 10% by mass, preferentially from 0.2% by mass to 5% by mass. Preferentially, the metals utilized are Mg, Ca, Ba, Sr, La, Ce, used individually or in mixtures thereof.

A third family of catalysts used comprises bifunctional catalysts, including:
a substrate, from the following list: MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, BETA, FAU, LTL, MOR, and the ZSM-48 family of microporous materials consisting of silicon, aluminum, oxygen, and optionally boron. MFI or MEL (Si/Al>25), MCM-41, MCM-48, SBA-15, SBA-16, SiO2, Al₂O₃, hydrotalcite, or a mixture thereof.
a metal phase (Me) amounting to 0.1% by mass, the metal being selected from the following elements: Zn, Mn, Co, Ni, Ga, Fe, Ti, Zr, Ge, Sn, and Cr used individually or as a mixture. These metal atoms may be inserted into the tetrahedral structure of the substrate. The incorporation of this metal may be done either by adding this metal during the synthesis of the substrate, or by incorporating it after synthesis by ion exchange or impregnation, with the metals thus then being incorporated in the form of cations, and not integrated within the structure of the substrate.

Prior to its use, the zeolite may be subjected to a variety of treatments, which may include: ion exchange; modification with metals; steaming; acid treatments or any other appropriate de-alumination method; mesoporization treatments; silicon surface passivation by silicon deposition; or any combination of the aforementioned treatments.

The content of alkalis or rare earths ranges from 0.05% by mass to 10% by mass, preferentially from 0.2% by mass to 5% by mass. Preferentially, the metals utilized are Mg, Ca, Ba, Sr, La, Ce, used individually or in mixtures thereof.

A fourth family of catalysts used includes amorphous solids such as silica-alumina, silica-phosphate, silica-borate, silica-titanium, silica-zirconia and/or mixtures thereof.

The catalyst can be a mixture of the materials described above in the four catalyst families. In addition, the active phases may also be combined with other constituents (binder, matrix) that impart greater mechanical strength or enhanced activity to the final catalyst.

If the hydrocarbon feedstock is oligomerized in a plant comprising a plurality of reactors in series, the reactors in the series may be fed with the same or different catalysts.

In one variant, the oligomerization step (c) for oligomerizing olefins is carried out in an oligomerization reactor, and the alkylation step (d) for alkylating aromatics is carried out in an alkylation reactor, separately from the oligomerization step (c) for oligomerizing olefins.

Advantageously, the water-depleted mixture is then separated in the first column into the C1-C2 hydrocarbon fraction, for example taken from the top of the column; into a C3-C5 hydrocarbon fraction, for example withdrawn from an intermediate stage of the column; and into a C6+ hydrocarbon fraction, for example taken from the bottom of the column. The C1-C2 hydrocarbon fraction and the C6+ hydrocarbon fraction are sent to alkylation step (d) in the alkylation reactor, while the C3-C5 hydrocarbon fraction is sent to oligomerization step (c) in the oligomerization reactor.

By way of a variant, the water-depleted mixture is separated into a C3 hydrocarbon fraction, for example taken from the top of the column; into a C4-C5 hydrocarbon fraction, for example withdrawn from an intermediate stage of the column; and into a C6+ hydrocarbon fraction, for example taken from the bottom of the column. The C3− hydrocarbon fraction and the C6+ hydrocarbon fraction are at least partially sent to alkylation step (d) in the alkylation reactor, while the C4-C5 hydrocarbon fraction is at least partially sent to oligomerization step (c) in the oligomerization reactor.

The product from the oligomerization reactor contains more than 50% by mass of C7+ olefins, in particular more than 60% by mass of C9 to C12 olefins.

The alkylation takes place under temperature and pressure conditions that are effective for maintaining in liquid phase more than 20% by mass of the feedstock in the alkylation zone.

Advantageously, the alkylation of aromatics with alkenes is carried out in the liquid phase, the aromatics being present essentially in liquid phase. It may also be effected using solid acid catalysts. Zeolites and silica-alumina catalysts having shape selectivity are generally used.

In this process, the reactor conditions are selected so as to ensure that the alkene introduced into the reactor is mostly dissolved in the aromatic feedstock. This is usually done by an optimal combination of operating conditions, such as pressure, temperature and choice of catalyst, with a sufficiently high catalytic activity. The gas-phase alkenes present can cause rapid deactivation of alkylation catalysts typically used in the liquid phase.

Examples of operating conditions that may be used are provided in U.S. Pat. No. 4,891,458, which describes the liquid-phase synthesis of ethylbenzene with zeolite beta, while U.S. Pat. No. 5,334,795 describes the use of MCM-22 in the liquid-phase synthesis of ethylbenzene; U.S. Pat. No. 7,649,122 describes the use of MCM-22 in the liquid-phase synthesis of ethylbenzene where the given water content level is maintained. U.S. Pat. No. 4,549,426 describes the liquid-phase synthesis of alkylbenzene with steam-stabilized zeolite Y; U.S. Pat. No. 8,134,036 describes the liquid-phase aromatic alkylation over at least one catalyst bed containing a first catalyst modified with the inclusion of a rare-earth metal ion.

The types of products that are able to be produced preferably correspond to the following generic chemical formulae: monoalkyl benzene, dialkyl benzene and trialkyl benzene. The alkyl chains (Rx) each have 2 to 10 carbon atoms, preferably 2 to 6 carbon atoms. These chains can be of equal or different lengths.

The aromatic compounds produced during conversion step (a) for converting C1 to C6 alcohols are typically mono-aromatics, optionally alkylated (benzene, toluene, ethylbenzene and xylenes), the alkylating agent being olefins.

The alkylation reaction is exothermic, with the consequence that it may be useful to inject a portion of the aromatics and/or a portion of the olefins between the different reactor beds, in the event of multiple beds. Aromatics having fewer than 8 carbon atoms may be recycled, as also olefins that are too short, for example having fewer than 5 carbon atoms.

The alkylation catalyst is for example in bead form, but more often in extruded form. It is made up of an acidic solid mixed with an amorphous phase. The forming of the acidic solid is effected by means of a matrix, which is an amorphous phase. The acidic solid is preferably at least one zeolite, preferably selected from zeolites of FAU structural type and more particularly zeolite Y, zeolites of MOR structural type (i.e. mordenite zeolite), zeolites of EUO structural type, (i.e. zeolites EU-1, ZSM-50, TPZ-3), zeolite NU-87 of NES structural type, zeolite NU-86 (described in EP 463 768 A), zeolite NU-85 (described in EP 462 745 A), zeolite NU-88 (described in FR 2 752 567), and zeolite IM-5 (described in FR 2 754 809), zeolite Beta, zeolite MCM-22, zeolite MCM-36, zeolite MCM-49, or zeolite MCM-56.

Preferably, the catalyst is a zeolite beta having a silica/alumina molar ratio (expressed as SiO2/Al2O3) from about 10 to about 200 or from about 20 to about 50.

Zeolite Beta may have a low sodium content, for example less than about 0.2% by mass expressed as Na2O, or less than about 0.02% by mass. The sodium content may be reduced by any method known to the skilled person, such as ion exchange.

These zeolites are at least partly in acid form (H+), but may also contain cations other than H+ such as alkaline earths or rare earths. The zeolitic catalyst may be modified with a rare earth metal ion, such as lanthanum, cerium, neodymium or praseodymium, for example.

The Brunauer-Emmett-Teller (BET) surface area of the catalyst used is between 50 m2/g and 900 m2/g, preferably between 100 m2/g and 700 m2/g. The Na/Al ratio of the final catalyst is less than 5 atomic % and preferably less than 2%.

The zeolite content in the catalyst is in particular between 5% by mass and 95% by mass, preferably between 10% by mass and 90% by mass, relative to the final catalyst. The overall Si/Al ratio of these zeolites is between 2.6 and 200, preferably between 5 and 100, and even more preferably between 5 and 80.

The matrix of the catalyst is a substrate selected from the group formed by alumina, silica, silica-alumina, alumina-boron oxide, magnesia, silica-magnesia, zirconia, titanium oxide and clay, these compounds being used individually or as mixtures thereof. It is preferred to use an alumina substrate.

Preferably the solid acid catalyst has a shape selectivity so as to avoid the formation of alkylaromatics of an excessively large size, such as those having more than 16 carbon atoms. If the molecular size of the alkylaromatics is close to the micropore size in the catalyst, formation and diffusion upon output from the pores is still feasible; on the other hand, the formation of alkylaromatics that are too large to enter, reside in or exit the pores thus does not occur.

As indicated above, the reaction zone is operated at a temperature and pressure such as to maintain phase conditions that preferably present more than 20% by mass of liquid.

For the production of alkylaromatics, having at least 8 carbon atoms, the reaction temperature is notably between 140° C. and 320° C., and is generally between 160° C. and 280° C. In one embodiment, the reaction temperature is between 190° C. and 240° C.

The alkylation pressure is generally maintained at sufficiently high levels to ensure the presence of a liquid phase. In one embodiment, the pressures are between 20 barg and 100 barg, in particular from 30 barg to 50 barg.

When operating under predominantly liquid phase conditions, an upflow reactor mode is generally used. The flow rates can typically vary from the liquid hourly space velocity (LHSV) between about 1 h−1 and 100 h−1 per bed, preferably between about 2 h−1 and 70 h−1 per bed. The aromatic/alkylating agent ratio is, for example, between 0.05 mol/mol and 20 mol/mol, and preferentially between 0.1 mol/mol and 10 mol/mol.

In one preferred mode of operation, the oligomerization of olefins and the alkylation of aromatics with olefins is carried out on the same catalyst and in the same reactor. The known operating conditions for oligomerization and alkylation are very similar and can easily be adapted in order to achieve the desired oligomerization and alkylation performance.

When oligomerization and alkylation are carried out simultaneously in the same reactor using the same catalyst, the reactor product contains more than 10% by mass of C8+ aromatics, in particular more than 6% by mass of C8 to C14 aromatics.

When alkylation in the presence of olefins is carried out separately from oligomerization, the reactor product contains more than 65% by mass of C8+ aromatics, in particular more than 75% by mass of C8 to C14 aromatics.

### Step (e) Formation of the Stream of Hydrocarbons to be Hydrogenated

A stream of hydrocarbons to be hydrogenated is formed from at least a portion of the olefins oligomerized in step (c) and at least a portion of the aromatics alkylated in step (d).

In the event that a single common stage is used for the olefin oligomerization step (c) and the aromatics alkylation step (d), the product from this stage is partially or totally used to form the stream of hydrocarbons to be hydrogenated.

This product comprises, for example, on a dry basis
less than 15% by mass of paraffins, in particular between 3% and 10% by mass of paraffins,
less than 10% by mass of C4 to C7 olefins, in particular between 0.5% and 5% of C4 to C7 olefins,
more than 50% by mass of C8 to C16 olefins, in particular between 60% and 80% of C4 to C16 olefins,
less than 5% by mass of C17+ olefins, in particular between 0.1% by mass and 1.0% by mass of C17+ olefins; and/or
less than 5% of C6-C7 aromatics, in particular between 0.5% and 4.0% of C6-C7 aromatics,
more than 2% of C8+ aromatics, in particular between 6% and 30% of C8+ aromatics.

In one variant, an optional separation step for separating the effluent containing at least a portion of the oligomerized olefins from step (c) and/or at least a portion of the alkylated aromatics from step (d) is carried out in an additional distillation column.

The separation produces at the column top a fraction of C7− hydrocarbons, and at the bottom a fraction of C8+ hydrocarbons.

The C7− hydrocarbon fraction preferably contains more than 90% by mass, in particular more than 95% by mass, of the C7− hydrocarbons contained in the effluent.

The C8+ hydrocarbon fraction comprises more than 90% by mass of the C8+ hydrocarbons contained in the effluent.

Advantageously, at least a portion, for example less than 50% by mass, of the C7-hydrocarbon fraction is at least partially recycled in the olefin oligomerization step (c) and/or in the aromatic alkylation step (d), with another portion forming a gasoline stream.

The stream of hydrocarbons to be hydrogenated is formed by at least a portion, preferably by the totality, of the C8+ hydrocarbon fraction.

In the case where the oligomerization step (c) for oligomerizing olefins is carried out in an oligomerization reactor, and the alkylation step (d) for alkylating aromatics is carried out in an alkylation reactor, separately from the oligomerization step (c) for oligomerizing olefins, the oligomerization reactor product and the alkylation reactor product containing the alkylated aromatics advantageously undergo a separation.

In one embodiment, the oligomerization reactor product containing oligomerized olefins and the alkylation reactor product containing alkylated aromatics are separated, at the bottom, into the C8+ hydrocarbon fraction and, at the top, into the C7− hydrocarbon fraction. The C7− hydrocarbon fraction is recycled at least partially (for example, less than 50% by mass) in step (c) in the oligomerization reactor.

At least a portion, preferably the totality, of the C8+ hydrocarbon fraction forms the stream of hydrocarbons to be hydrogenated.

In one other embodiment, the product from the oligomerization reactor and the product from the alkylation reactor are separated in the distillation column into a C7-hydrocarbon fraction, withdrawn at the top; into a C8 to C16 hydrocarbon fraction, taken from an intermediate stage; and into a C17+ hydrocarbon fraction, withdrawn at the bottom.

At least a portion, preferably the totality, of the C8 to C16 hydrocarbon fraction forms the stream of hydrocarbons to be hydrogenated.

The C17+ hydrocarbon fraction is recycled at least partially in conversion step (a) for converting the C1 to C6 alcohol stream. This recycling is carried out in order to once again crack the C17+ olefins.

### Step (f) Hydrogenation

The stream of hydrocarbons to be hydrogenated undergoes hydrogenation so as to form a stream of hydrogenated hydrocarbons. This saturates the olefinic compounds and partially hydrogenates the aromatic compounds.

Hydrogenation is carried out, for example, in one or more mixed-phase, fixed-bed (descending or ascending) reactors, with the fraction to be hydrogenated being predominantly in the liquid phase.

The hydrogenation is carried out at a temperature of between 50° C. and 350° C., in particular between 100° C. and 300° C. It is performed with the pressure being preferably greater than 10 bara, and in particular between 20 bara and 80 bara.

A stream of hydrogen is fed into the or each reactor in admixture with the hydrocarbon stream to be treated. The ratio of the volume flow rate of the hydrogen stream to the volume flow rate of the hydrocarbon stream (excluding the recycle stream) to be hydrogenated is advantageously between 50 NL/L and 3000NL/L, in particular between 100 NL/L and 500 NL/L. The hydrogen may be added to the hydrocarbon stream in several stages along the catalytic bed. The hourly space velocity is advantageously between 0.5 and 3 and in particular between 1 and 2 hW. Excess hydrogen may be recycled back into the reaction zone after separation and compression.

The reaction is carried out in the presence of at least one catalyst comprising one or more Group VIII metals (typically Pt, Pd, Ni) supported on a substrate such as silica, alumina, or any suitable mixture of these two compounds, or carbon. The reaction can also be carried out in the presence of a sulfide catalyst containing a Group VIB element (Cr, Mo, W) and a Group VIIIB element (Fe, Ru, Co, Os, Co, Rh, Ir, Pd, Ni, Pt) or mixtures of these two metal groups.

The hydrogenated hydrocarbon stream advantageously contains less than 10% by mass of olefins and preferably less than 3% of olefins.

It preferably contains more than 50% by mass of paraffins, in particular more than 50% by mass of C7 to C17 paraffins, in particular more than 60% by mass of C7 to C17 paraffins, in particular between 70% by mass and 95% by mass of C7 to C17 paraffins.

According to one variant between 10% and 90% by mass of the aromatics contained in the stream of hydrocarbons to be hydrogenated, preferably between 30% by mass and 80% by mass of the aromatics contained in the stream of hydrocarbons to be hydrogenated are hydrogenated into cycloparaffins.

The stream of hydrogenated hydrocarbons includes, for example:
more than 20% by mass of C9 hydrocarbons, for example between 20% and 40% by mass of C9 hydrocarbons;
more than 20% by mass, for example between 20% by mass and 40% by mass of C12 hydrocarbons;
more than 6% by mass of C6+ aromatics, in particular more than 6% by mass of C8+ hydrocarbons.

### Step (g) Recovery of the Jet Fuel Fraction

In order to recover the jet fuel fraction, the stream of hydrogenated hydrocarbons advantageously undergoes fractionation. This fractionation may be carried out by passing the stream through at least one separation column, such as a distillation column.

Preferably, at least one first separation column is used to separate, at the top, a liquefied petroleum gas fraction from the rest of the hydrogenated hydrocarbon stream that is obtained at the bottom.

The fractionation conditions in this column are as follows: pressure advantageously between 2 bara and 15 bara, column top condensation temperature adjusted so as to be able to use an air or cooling water condenser, that is to say preferably between 20° C. and 50° C.

The residual fraction of the hydrogenated hydrocarbon stream is then introduced into a second separation column in order to produce a naphtha fraction at the top, a diesel fraction at the bottom, and a jet fuel fraction at least one intermediate stage. This fractionation may be carried out in a single column, comprising a lateral draw-off of the jet fuel fraction, or in two separate columns.

More than 80% by mass of the stream of hydrogenated hydrocarbons introduced into the second column advantageously forms the jet fuel fraction.

The fraction of liquefied petroleum gases preferentially has a final boiling point of less than 180° C., and even more preferentially a final boiling point of less than 150° C.

The initial boiling point may range from 20° C. to 60° C. and preferably from 25° C. to 40° C.

The naphtha fraction comprises more than 80% by mass of the C8− paraffins contained in the residual fraction.

The jet fuel fraction contains between 2% by volume and 30% by volume of C8+ aromatics, preferably between 6% by volume and 25% by volume of C8+ aromatics, and even more preferably between 8% by volume and 25% by volume of C8+ aromatics.

It contains more than 50% by volume of C9 to C16 paraffins, in particular between 60% by volume and 95% by volume of C9 to C16 paraffins.

In particular, the jet fuel fraction contains more than 60% by volume of C9 to C12 paraffins.

It preferentially has a final boiling point of less than 400° C., and even more preferentially a final boiling point of less than 350° C.

The initial boiling point may range from 130° C. to 180° C.

The diesel fraction is the heaviest fraction, whereof not all of the molecules can be used to form jet fuel. The initial boiling temperature of this fraction is typically above 300° C., preferentially being 310° C.

Advantageously, at least a portion of the diesel fraction is recycled to conversion step (a) for converting the C1 to C6 alcohol stream in the form of a recycle stream in order to generate further cracking of the compounds present in this fraction.

The recycle stream advantageously constitutes between 10% by mass and 50% by mass of the C1 to C6 alcohol stream introduced in the conversion step (a).

### BRIEF DESCRIPTION OF THE DRAWINGS

The invention will be better understood upon reading the following description, provided only by way of example, and made with reference to the annexed drawings, wherein:

FIG. 1 is a schematic view of a plant configured for the implementation of a first fuel production process for producing a jet fuel according to the invention;

FIGS. 2 to 7 are views similar to that in FIG. 1 illustrating variants of the plant designed for implementation of the variants of the process shown in FIG. 1:

FIG. 8 is a detail view illustrating a reactor for implementing the fluidized bed-based conversion step;

FIG. 9 is an NMR spectrum of a ZSM5 parent catalyst and a modified catalyst having an ALPO structure.

FIG. 10 illustrates the selectivities obtained when implementing an exemplary conversion step of the process according to the invention;

FIG. 11 illustrates the conversion of certain olefins as a function of time during an exemplary implementation of the oligomerization step with a first catalyst;

FIG. 12 illustrates the conversion of certain olefins as a function of time during an exemplary implementation of the oligomerization step with a second catalyst;

FIG. 13 illustrates the conversion of certain aromatics as a function of time during an exemplary implementation of the aromatics alkylation step with the second catalyst.

### DETAILED DESCRIPTION

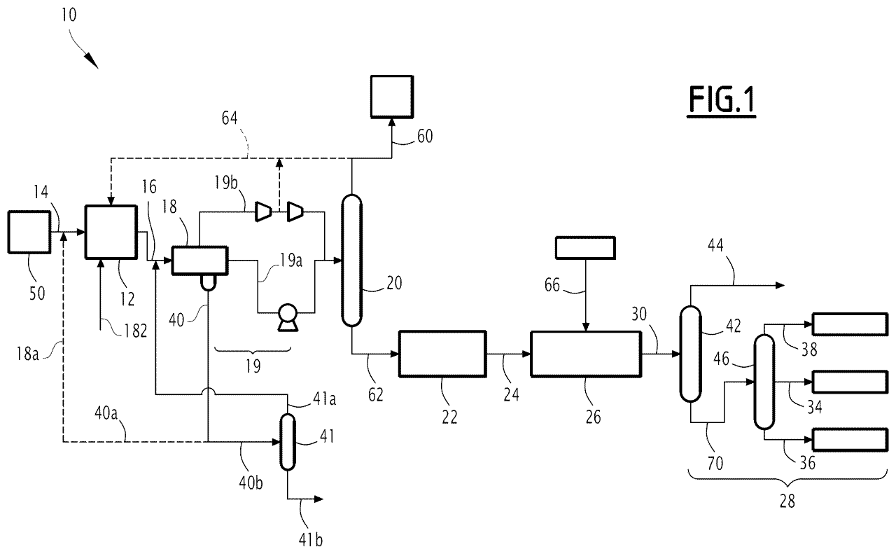

A first production plant 10, designed to implement a jet fuel production process according to the invention, is illustrated schematically in FIG. 1.

The first plant 10 comprises a conversion stage 12 for converting a C1 to C6 alcohol stream 14, intended to produce a mixture 16 containing paraffins, olefins, aromatics, and water; and a separation stage 18 for separating water from the mixture 16 in order to produce a water-depleted mixture 19 comprising a liquid phase 19a and a gas phase 19b. The plant shown in FIG. 1 also includes a separation stage 20 for separating the C1 to C2 hydrocarbons out of the water-depleted mixture.

In this example, the plant 10 additionally comprises a joint oligomerization and alkylation stage 22 for oligomerizing olefins and alkylating aromatics originating from the water-depleted mixture, with the stage 22 producing a stream 24 of hydrocarbons to be hydrogenated.

The plant 10 further comprises a hydrogenation stage 26 for hydrogenating the stream 24, thereby producing a stream of hydrogenated hydrocarbons 30; and a fractionation stage 28 for fractionating the stream of hydrogenated hydrocarbons 30, which is designed to fractionate at least one jet fuel fraction 34, and advantageously one diesel fraction 36 and one naphtha fraction 38.

The conversion stage 12 is designed to implement the conversion step (a) described above, which transforms the C1 to C6 alcohols predominantly into C3 to C7 olefins.

As described above, the conversion stage 12 comprises for example, at least one fixed-bed reactor, for example a plurality of fixed-bed reactors, with the one or more fixed-bed reactor(s) advantageously defining a plurality of successive catalytic fixed beds, as described above.

According to the invention, the conversion stage 12 comprises at least one supply conduit for supplying a carbon dioxide-containing stream 182, with the conversion stage 12 for converting the C1 to C6 alcohol stream 14 being configured so as to convert the carbon dioxide deriving from the carbon dioxide-containing stream 182 into carbon monoxide jointly with the conversion of the C1 to C6 alcohol stream.

The supply conduit opens, for example, upstream of the one or more reactor(s) of the conversion stage 12, in order for the carbon dioxide-containing stream 182 to mix with the C1 to C6 alcohol stream 14, or/and in one of the reactors of the conversion stage 12 or/and between two reactors of the conversion stage 12.

With reference to the above description, the separation stage 18 is designed to implement the separation step (b) for separating water. It comprises at least one separator that operates by gravity and/or by mechanical drive in order to separate the water out of the mixture 16, thereby making it possible to recover a water-concentrated aqueous fraction (stream 40), a gas-phase hydrocarbon fraction 19b, and a liquid-phase hydrocarbon fraction 19a.

Optionally, the plant 10 includes at least one recycle conduit 18a for recycling water separated in the separation stage to the conversion stage 12. The recycle conduit 18a opens, for example, upstream of the fixed catalytic beds or between two successive fixed catalytic beds in the event of a conversion stage 12 comprising at least one fixed-bed reactor.

Advantageously, the separation stage 18 includes a stripping column 41 that is capable of treating at least a portion of the separated water that forms the stream 40 so as to extract the hydrocarbons that it contains and obtain treated water.

The plant 10 comprises at least one cooling device (e.g. a heat exchanger) that serves to reduce the temperature of the product being output from the reactor and heating another stream, such as the feed stock to the reactor 12; a water- or air-cooled cooler; and/or a combination of the foregoing downstream of the conversion stage 12 in order to condense the water and produce the stream 40.

The separation stage 20 is designed to implement separation step (c) for separating lighter hydrocarbons than C3 hydrocarbons, such as C1-C2 hydrocarbons, and light compounds such as CO, CO2 and hydrogen, as defined above. Advantageously, it comprises at least one de-ethanizer. As the separation stage 20 operates at a higher pressure than the water separation stage 18, the plant comprises at least one pump which is capable of increasing the pressure of the liquid phase 19a and at least one compressor which is capable of increasing the pressure of the gas phase 19b.

The olefin oligomerization and aromatics alkylation stage 22 is designed to jointly implement steps (d) and (e). It comprises at least one joint oligomerization and/or alkylation reactor designed to implement the experimental conditions as described above.

The hydrogenation stage 26 is designed to implement step (f). It comprises at least one fixed-bed hydrogenation reactor designed to carry out the hydrogenation reaction under the conditions described above.

The fractionation stage 28 is designed to implement step (g). In this example, it comprises at least a first upstream separation column 42 for separating liquefied petroleum gas 44, and a second downstream fractionation column 46, designed to produce fractions 34 to 38.

A first example of a jet fuel production process, implemented in the plant shown in FIG. 1, will be described below.

Initially, a C1 to C6 alcohol stream 14 is conveyed to the conversion stage 12. The alcohol stream 14 originates, for example, from a source 50 as described above, in which the alcohols from the source 50 are produced, for example, by fermentation of biomass, catalytic conversion of carbohydrates or carbon monoxide or carbon dioxide, in the presence of hydrogen.

The stream 14 comprises of the composition described above, for example with more than 50% by dry weight of methanol and preferably more than 80% by dry weight of methanol.

The stream 14 is introduced into the conversion stage 12 where it undergoes a conversion described above comprising dehydration/aromatization of the C2 to C6 alcohols, and in the case of methanol, conversion into dimethyl ether followed by dehydration.

The reaction is carried out under the operating conditions in terms of temperature and pressure as described above. One or more of the catalysts defined above are used.

According to the invention, a stream containing carbon dioxide 182 is added to the conversion stage 12.

The carbon dioxide stream 182 comprises more than 5% by mass of carbon dioxide as defined above.

In the feedstock supplied to the conversion stage 12, the mass ratio of carbon dioxide to C1-C6 alcohols is between 5% and 75%.

The carbon dioxide contained in the carbon dioxide stream 182 is converted into carbon monoxide in conjunction with the conversion of the C1 to C6 alcohol stream.

A mixture 16 containing paraffins (in particular n-paraffins, i-paraffins and cyclo-paraffins), olefins, aromatics, and water is thus obtained. For example, the mixture 16 comprises of the composition described above.

The mixture 16 is then introduced into the separation stage 18 in order to produce a water stream 40 at the bottom of the separator, as well as the water-depleted mixture 19 comprising the gas phase 19b and the liquid phase 19a.

A portion 40a of the water stream 40 is optionally recycled to the conversion stage 12, via the conduit 18a, as described above. Another portion 40b of the water stream 40 is introduced into a column 41 so as to undergo stripping and to produce, at the top, a stream 41a of extracted hydrocarbons and, at the bottom, a treated water stream 40b that has a lower hydrocarbon content than that of the water stream 40.

The extracted hydrocarbon stream 41a is recycled to the separation stage 18, for example upstream of the separator.

The gas phase 19b and liquid phase 19a are then introduced, after compression, into the deethanizer in the separation stage 20. The deethanizer operates under the conditions defined above and produces at the top, a fraction 60 of C1-C2 hydrocarbons that may contain light compounds such as CO, CO2, hydrogen; and at the bottom, a fraction 62 of C3+ hydrocarbons.

The fractions 60 and 62 obtained have the compositions defined above.

Preferably, the C1-C2 hydrocarbon fraction 60, optionally after separation, is sent into a steam cracker for recovery of at least a portion of the ethylene contained therein.

In one variant represented as a dotted line in FIG. 1, at least a portion 64 of the C1 to C2 hydrocarbon fraction 60 is recycled in the separation stage 12 in the form of a recycle stream 64. Optionally, at least a fraction of the gas phase 19b, for example less than 50% by volume of the gas phase 19b, is also recycled, without passing through the deethanizer.

The ratio of the mass flow rate of the recycle stream 64 to the mass flow rate of the fraction 60 output from the top of the deethanizer of the separation stage 20 is less than 0.5 as defined above.

In one variant, the carbon dioxide, carbon monoxide and hydrogen present in the C1-C2 hydrocarbon fraction are separated from the other hydrocarbons, in particular by cryogenic distillation, membrane separation, or pressure swing adsorption and combinations thereof.

These compounds are then advantageously recycled to a preliminary synthesis step for synthesizing alcohols, in particular by synthesis gas fermentation and by catalytic conversion of the synthesis gas in order to produce in particular methanol.

The methanol or ethanol thus produced is then advantageously recycled so as to form a portion of the C1 to C6 alcohol stream 12.

In the example shown in FIG. 1, the C3+ hydrocarbon fraction 62, having the composition defined above, is then introduced into the joint oligomerization and alkylation stage 22.

In this stage 22, one or more joint oligomerization and alkylation reactors carry out oligomerization of the olefins present in the fraction 62, according to the operating conditions defined above in the description, in particular oligomerization of C3 to C7 olefins.

In addition, in a joint manner, the C6+ aromatics present in the fraction 62 are alkylated to form, in particular, C8+ aromatics.

The reaction is carried out under the operating conditions described above. One or more of the catalysts defined above are used.

At the outlet of the stage 22, a stream 24 of hydrocarbons to be hydrogenated is formed, with the composition as defined above.

Thereafter, the stream 24 of hydrocarbons to be hydrogenated is introduced into the hydrogenation stage 26, in order to induce the hydrogenation of at least a portion of the olefins present in the stream of hydrocarbons to be hydrogenated 24, as well as the hydrogenation into cycloparaffins of at least a portion of the aromatics present in the stream of hydrocarbons to be hydrogenated 24.

The hydrogenation is carried out under the operating conditions described above, using one or more of the catalysts described above.

A hydrogen-containing stream 66 is introduced into the hydrogenation stage 26, with a ratio of the volume flow rate of hydrogen in the stream 66 to the volume flow rate of the stream of hydrocarbons to be hydrogenated 24 being, for example, as defined above.

A stream of hydrogenated hydrocarbons 30 is formed at the outlet of the hydrogenation stage with the composition described above.

The stream of hydrogenated hydrocarbons 30 is then fractionated in the fractionation stage 28.

In the first column 42, it is separated into a C4− hydrocarbon fraction that forms the liquefied petroleum gas fraction 44, and a C4+ hydrocarbon fraction that forms the residual fraction 70 of the stream of hydrogenated hydrocarbons.

The fractions 44, 70 have the characteristics defined above in terms of cut points.

The residual fraction 70 is introduced into the second column 46 so as to be fractionated therein into the naphtha fraction 38, the jet fuel fraction 34, and the diesel fraction 36, as characterized above.

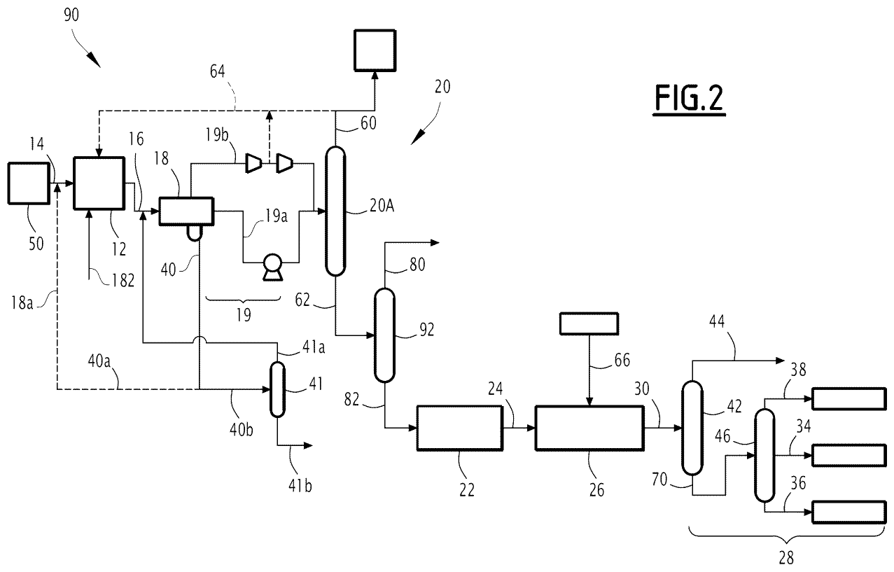

The plant variant 90 illustrated in FIG. 2 is designed to implement a second process according to the invention. It differs from the plant 10 illustrated in FIG. 1 in that the separation stage 20 comprises an additional recovery column 92 for recovering propylene.

The C3+ hydrocarbon fraction 62 obtained from the deethanizer of the separation stage 20 is introduced into the additional column 92 in order to form at the top of the column, a C3− hydrocarbon fraction 80, and at the bottom of the column, a C4+ hydrocarbon fraction 82, intended to be introduced into the joint oligomerization and alkylation stage 22.

The fractions 80, 82 have the compositions previously described above. The fraction 80 contains more than 80% by mass of the propylene contained in the C3+ hydrocarbon fraction 62. Such an example provides the means for recovering the propylene formed in the conversion stage 12, where such recovery is economically attractive.

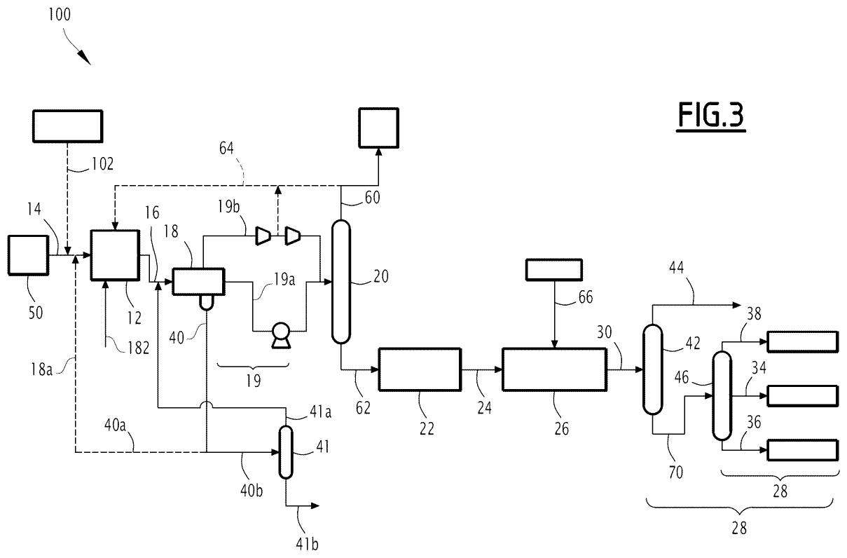

The plant 100 described in FIG. 3 is designed for implementing a third process according to the invention. It differs from the plant 10 described in FIG. 1 in that at least one tapping 102 for the adding of C2 to C6 alcohols is provided for in quenching in the conversion stage 12, for example between two successive catalytic beds of the conversion stage 12.

The composition of the C2-C6 alcohol stream 102 advantageously comprises less than 20% of methanol, and more than 80% of C2-C6 alcohol by mass, for example more than 50% of ethanol and propanol.

The adding of C2-C6 alcohols in addition to methanol facilitates the conversion reaction for converting the alcohol stream 14 by rendering it more isothermal (the conversion of methanol being highly exothermic and the conversion of C2-C6 alcohols being endothermic) and therefore easier to control, in particular when fixed catalytic beds are used in the conversion stage 12.

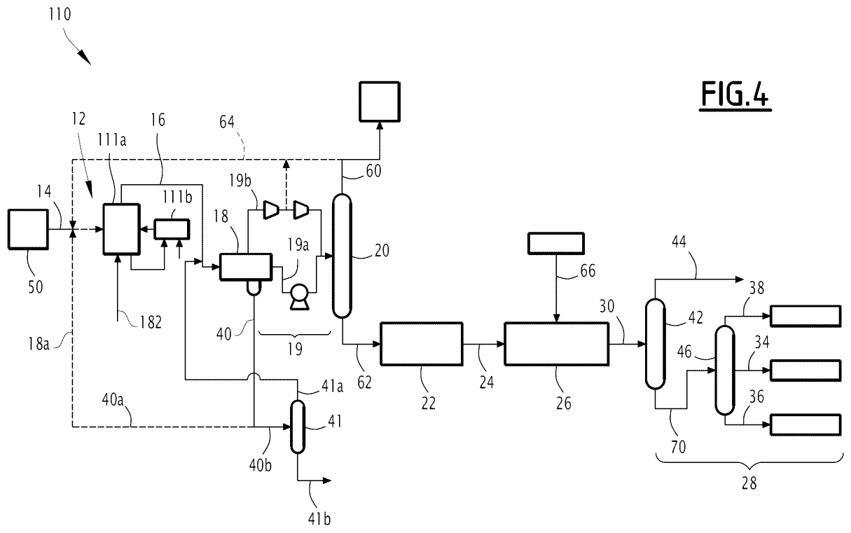

The plant 110 described in FIG. 4 is analogous to that shown in FIG. 1. It comprises a conversion stage 12 that has at least one reactor having a fluidized catalytic bed, preferably having a single fluidized catalytic bed reactor. Said one or more reactor(s) is/are capable of implementing the experimental conditions as described above.

The reactor comprises a reaction zone 111a having a fluidized catalytic bed, and a regeneration zone 111b for regenerating the fluidized catalytic bed. A portion of the catalyst present in reaction zone 111a is continuously withdrawn so as to be regenerated in the regeneration zone 111b, advantageously by controlled combustion in the presence of oxygen.

A portion 40a of the water stream 40 is optionally recycled to the conversion stage 12, via the conduit 18a.

Eventually, as an option, a portion 64 of the C1-C2 hydrocarbon fraction separated from the water-depleted mixture 19 is introduced into the reactor having a fluidized catalytic bed.

Advantageously, the C1 to C6 alcohol stream is introduced in conversion step (a) at a temperature at least 5° C. higher than the bubble point of the C1 to C6 alcohol stream.

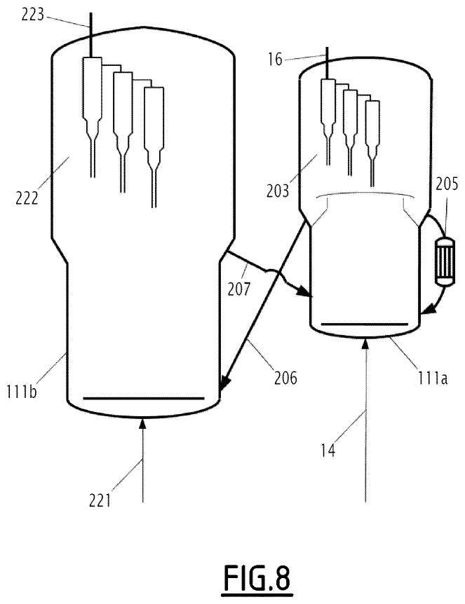

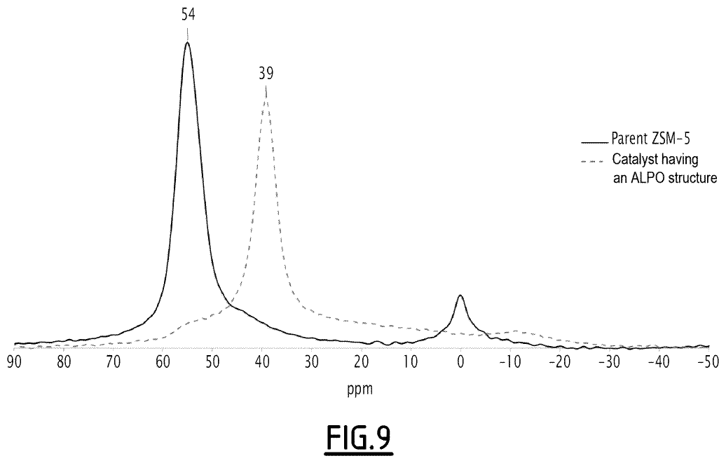

FIG. 8 illustrates, in one particular embodiment, the flow of catalyst from the regeneration zone 111b to the reaction zone 111a.

For reasons of simplicity, the drawings do not include details of the internal parts of the vessels that form the zones 111a, 111b.

The C1 to C6 alcohol stream 14 is introduced into the bottom of the reaction zone 111a, which features a fluidized catalytic bed.

At the top of the reaction zone 111a, the products from the conversion reaction are separated from the catalyst in a disengagement zone 203, advantageously equipped with cyclones, and the mixture 16 produced is conveyed to the separation stage 18.

Optionally, the reaction heat produced by the conversion is extracted from the reaction zone 111a by means of a catalyst cooler 205, which is advantageously a heat exchanger that is located outside the reaction zone 111a and connected to the latter.

The reaction zone 111a receives the catalyst regenerated in the regeneration zone 111b via a feed line 207 connecting the regeneration zone 111b to the reaction zone 111a.

The deactivated catalyst is removed from the disengagement zone 203 via a discharge line 206 that is separate from the feed line 207, with the discharge line 206 connecting the reaction zone 111a to the regeneration zone 111b.

Air is injected through the injection conduit 221 into the regeneration zone 111b, at the bottom of the latter, in a fluidized bed where coke deposits are burned off.

The regeneration zone 111b also comprises a disengagement zone 222, advantageously equipped with cyclones. In this zone 222, the flue gases are separated from the regenerated catalyst and discharged via the regeneration conduit 223, which is advantageously located at the top of the regeneration zone 111b.

Optionally, as the combustion of coke deposits is a highly exothermic reaction and the temperature in the regeneration zone 111b needs to be carefully controlled, a catalyst cooler (not represented in the figure, but similar to the catalyst cooler 205) is connected to the regeneration zone 111b. The hot catalyst extracted from the regeneration zone circulates through this cooler in order to be cooled, which thereby controls the temperature in the regeneration zone 111b. The regenerated catalyst is sent via line 207 to the reaction zone 111a.

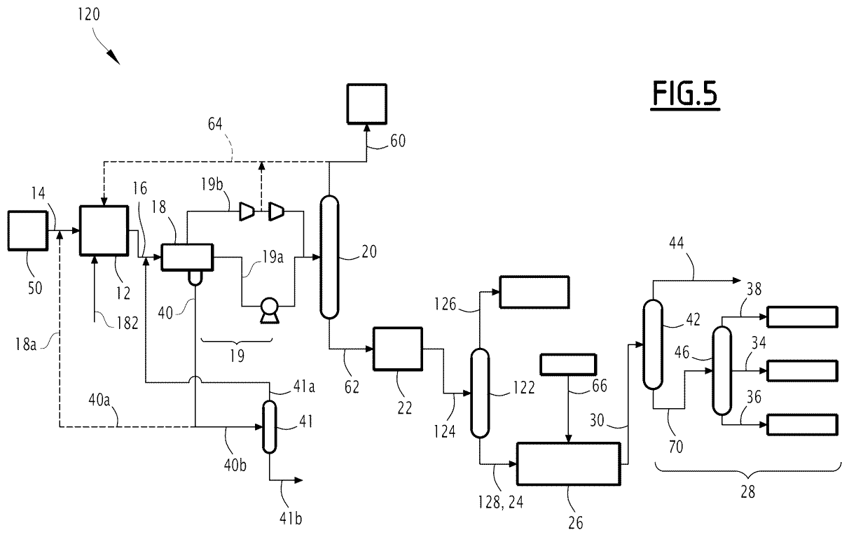

A fifth plant 120 designed for implementing a fifth process according to the invention is illustrated in FIG. 5. The fifth plant 120 differs from the first plant 10 in that it includes an additional separation stage 122 interposed between the outlet of the joint oligomerization and alkylation stage 22 and the inlet of the hydrogenation stage 26.

The additional separation stage 122 comprises at least one distillation column.

The product 124 obtained from the joint oligomerization and alkylation stage 22 is separated in the distillation column into a C7− hydrocarbon fraction 126 and a C8+ hydrocarbon fraction 128, which form the stream of hydrocarbons 24 destined to be hydrogenated. The C7− hydrocarbon fraction 126 may optionally be recycled to the joint stage 22.

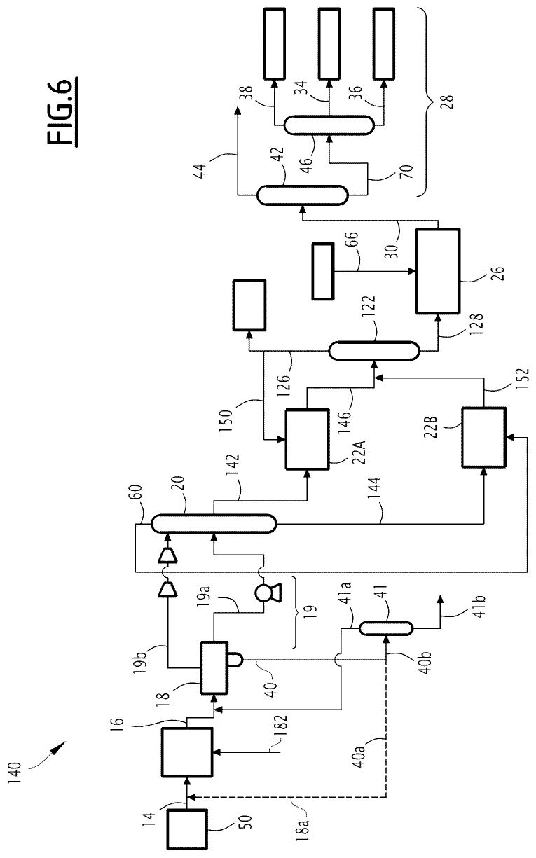

A sixth plant 140 according to the invention is illustrated in FIG. 6. This sixth plant 140 is designed for implementing a sixth process according to the invention. It differs from the first plant 10 in that it has a dedicated oligomerization stage 22A for oligomerizing the olefins deriving from the water-depleted mixture 19 coming from stage 20, and a dedicated alkylation stage 22B for alkylating the aromatics deriving from the water-depleted mixture 19 coming from stage 20.

Each stage 22A 22B comprises respectively, a separate oligomerization reactor and an alkylation reactor, wherein the operating conditions provided for above in the description are implemented.

In the separation stage 20, the water-depleted mixture 19 is separated into a fraction 60 of C1-C2 hydrocarbons recovered at the top of the deethanizer; a fraction 142 of C3 to C5 hydrocarbons recovered at an intermediate stage of the deethanizer; and a fraction 144 of C6+ hydrocarbons recovered at the bottom of the deethanizer.

The C3-C5 hydrocarbon fraction 142 is sent in its entirety to the oligomerization stage 22A in order to produce an oligomerization reactor product 146.

The C1-C2 hydrocarbon fraction 60 and the C6+ hydrocarbon fraction 144 are conveyed to the alkylation stage 22B in order to produce an alkylation reactor product 152 under the operating conditions defined above.

The product 152 from the alkylation reactor is then mixed with the product 146 from the oligomerization reactor.

The products 146, 152 are then introduced into the additional separation stage 122 so as to be separated into the C7− hydrocarbon fraction 126 and the C8+ hydrocarbon fraction 128, described above.

At least a portion 150 of the C7− hydrocarbon fraction 126 is recycled into the oligomerization stage 22A, with another portion possibly being recovered in the form of gasoline.

The fraction 128 forms the stream of hydrocarbons to be hydrogenated 24.

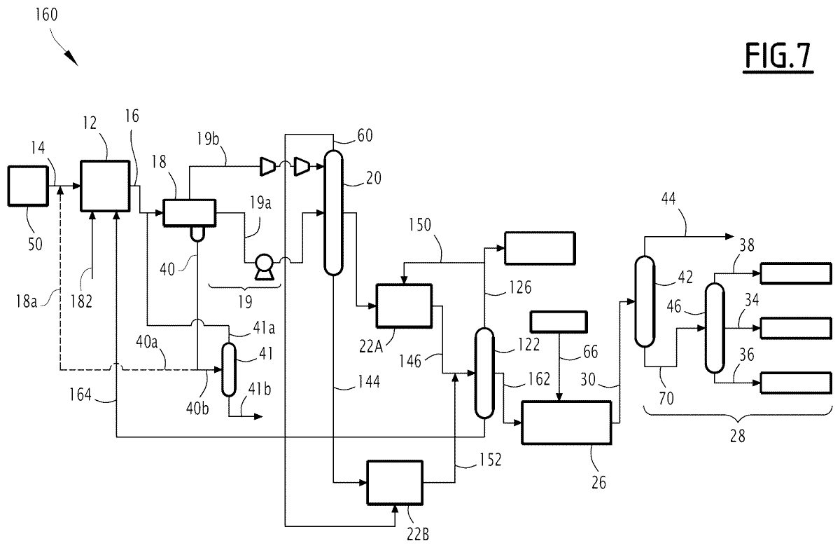

A seventh plant 160 designed for implementing a seventh process according to the invention is shown in FIG. 7.

The seventh process according to the invention differs from the sixth process implemented in the plant 150 in that, in the additional separation stage 122, the product 146 from the oligomerization reactor and the product 152 from the alkylation reactor are separated into the C7− hydrocarbon fraction 126, taken from the top of the column; a C8 to C16 hydrocarbon fraction 162, taken from an intermediate stage of the column; and a C17+ hydrocarbon fraction 164, taken from the bottom of the column.

As previously described above, at least a portion 150 of the C7− hydrocarbon fraction 126 is recycled back to the oligomerization stage 22A.

The C8 to C16 hydrocarbon fraction 162 is introduced into the hydrogenation stage 26 in order to be hydrogenated.

The C17+ hydrocarbon fraction 164 is at least partially recycled to the conversion stage 12.

Thus, the heavy hydrocarbons present in the C17+ hydrocarbon fraction are re-cracked in the conversion stage 12. This results in an increase in the amount of jet fuel fraction 34 produced.

In all the cases previously described above, the jet fuel fraction 34 produced by the above-mentioned processes can be utilized as such, in a pure manner, as an aircraft jet fuel intended for use in propelling an aircraft engine, or in a mixture blended with a jet fuel derived from the distillation of petroleum. The jet fuel fraction or blend thereof is advantageously a Sustainable Aviation Fuel (SAF), the composition of which is similar to the SAFs described according to the standard ASTM D7566.

The blend comprises at least 5% by mass, and in particular at least 10% by mass, of the jet fuel fraction 34.

Thanks to the invention as described above, it is possible to provide simple and efficient fuel production processes for producing a jet fuel from a stream of C1 to C6 alcohols, that is preferably from renewable sources, in particular deriving from fermentation, or/and generated by converting carbon monoxide or carbon dioxide captured from the atmosphere in the presence of hydrogen.

The jet fuel fraction produced by the process according to the invention has a very low carbon footprint, since it is not derived from petroleum derivatives, but on the contrary from sources that contribute to reducing the amount of carbon dioxide present in the atmosphere.

The jet fuel fraction 34 produced by the process according to the invention is moreover manufactured in a very economical manner and can in certain cases be used as such, as an aircraft engine propulsion fuel, without requiring further purification or without blending.

Naturally, the plants shown in FIGS. 2, 3, and 5 to 7 may also feature a conversion stage 12 equipped with a fluidized catalytic bed, as shown in FIG. 4.

In one variant, the process is implemented in order to produce olefins (in particular ethylene, propylene, butenes, pentenes and hexenes), while minimizing the production of paraffins from renewable resources, based on the conversion of a C1 to C6 alcohol stream into olefins.

### EXAMPLES

Some particular, non-limiting examples of implementation of the conversion step (a), the joint oligomerization and alkylation steps (c) and (d), and the hydrogenation step (f) will be described hereinafter.

### Step (a) Conversion

### Preparation of Catalysts

A sample of zeolite ZSM-5 (Si/Al=12) in H form (containing 445 ppm of Na, less than 25 ppm of K, 178 ppm of Fe, 17 ppm of Ca, and synthesized without matrix) was steam-treated at 550° C. for a period of 6 h in 100% H2O at atmospheric pressure. The sample is hereinafter identified as Sample A.

The steamed solid A was subjected to contacting with a 3.14 M solution of H3PO4 for a period of 4 h under reflux conditions (4.2 ml/g zeolite). Subsequently, solid A was then separated from the liquid phase at ambient temperature by filtration of the solution. The material obtained was dried at 200° C. for a period of 16 h. The sample is hereinafter identified as Sample B.

### Catalyst Example 1

490 g of Sample B was mixed with 490 g of specific binder (P=15.9 mass %, Si=13.2%, Mg=0.27%, Al=0.15 mass %, K=230 ppm, Na=230 ppm, Ca=19.2 mass %), 588.3 g of low-sodium silica sol containing 34 mass % of SiO2, 6 g of xonotlite, and 2 to 3 mass % of extrusion additives. The mixture was agitated for a period of 30 min and then extruded.

The specific binder was prepared by mixing the equivalent mass of NH4H2PO4 and xonotlite in aqueous medium at ambient temperature (1 g solid/4 ml water). After agitation for a period of 60 minutes, the phosphated xonotlite was separated from the liquid by filtration and dried. The dried product was used as an extrusion component.

The extruded solid was dried for a period of 24 h at ambient temperature, then 16 h at 200° C., followed by washing with demineralized water at ambient temperature and subsequent drying at 110° C. overnight. An additional step of washing at ambient temperature was then carried out using demineralized water at pH 3.08. The catalyst is then dried at 110° C. overnight and calcined at 700° C. for 2 hours.

### CATALYST EXAMPLE 2

320 g of Sample B were mixed with 400 g of specific binder (P=15.9 mass %, Si=13.2, Mg=0.27, Al=0.15 mass %, K=230 ppm, Na=230 ppm, Ca=19.2 mass %), 165 ml H2O, 235 g low-sodium silica sol containing 34 mass % of SiO2 and 2 to 3 mass % of extrusion additives. The mixture was agitated for a period of 30 min and then extruded.

The specific binder was prepared by mixing the equivalent mass of (NH4)H2PO4 (ammonium dihydrogen phosphate) and xonotlite in an aqueous medium at ambient temperature (1 g solid/4 ml water). After agitation for a period of 60 minutes, the phosphated xonotlite was separated from the liquid by filtration and dried. The dried product was used as an extrusion component.

The extruded solid was dried for a period of 24 h at ambient temperature, then 16 h at high temperature followed by washing and steam heat treatment at 600° C. for a period of 2 h. The sample is hereinafter identified as Sample E.

### Catalyst Example 3

356 g of Sample A was extruded with 338.7 g of Nyacol (40% by mass SiO2 sol), 311.3 g of fumed silica (FK500), 480 ml of H2O and 2 to 3% of extrusion additives. The extruded solid was dried for a period of 24 hours at ambient temperature, then 16 hours at 110° C. followed by calcinations at 500° C. for a period of 10 hours. The final sample contained 40% by mass of zeolite and 60% by mass of SiO2 binder. The extruded sample was subjected to ion exchange with 0.5 M NH4Cl under reflux conditions for a period of 18 hours followed by washing with water, drying at 110° C. for a period of 16 hours and calcinations at 450° C. for a period of 6 hours. The sample having undergone forming and ion exchange was treated with 3.1 M H3PO4 under reflux conditions for a period of 4 h (1 g/4.2 mL), followed by cooling, filtration and drying at 110° C. for a period of 16 h.

The phosphate sample was washed at ambient temperature with a 0.1 M solution of calcium acetate for a period of 2 h (1 g/4.2 ml). Then, the washed sample was dried at 110° C. for a period of 16 hours and steam heat treated in 100% by mass of H2O for a period of 2 hours at 600° C.

### CATALYST EXAMPLE 4

150 g of Sample B was subjected to contacting with 630 ml of aqueous solution containing 1.5 g of dispersed xonotlite, followed by the addition of 450 g of low-sodium silica sol (34 mass % SiO2 in water, 200 ppm Na). Thereafter, the solution was agitated for a period of 1 hour and spray-dried. The spray-dried solid was washed with water at ambient temperature for a period of 2 hours followed by filtration, drying at 110° C. for a period of 16 hours and calcinations at 700° C.

### CATALYST EXAMPLE 5

100 g of sample A was subjected to contacting with 25 g of 85% by mass H3PO4 under reflux conditions for a period of 4 h, followed by cooling and the addition of 120 ml of aqueous solution containing 7 g of dispersed xonotlite. The resulting slurry was kept under agitation for a period of around 1 h, followed by the addition of 300 g of low-sodium silica sol (34% by mass SiO2 in water, 200 ppm Na). Thereafter, the solution was agitated for a period of one hour and spray-dried. The spray-dried solid was dried at 200° C. for a period of 16 h and washed with water at ambient temperature for a period of 2 h, followed by filtration, drying and calcinations at 700° C. for a period of 2 h.

### CATALYST EXAMPLE 6

75 g of Sample A was introduced into a solution containing 14.25 g of 85 mass % H3PO4 and 300 ml of demineralized water. The suspension was agitated under reflux for a period of 2 h. Then 4.125 g CaCO3 were added to the suspension. Heating of the solution was stopped, while maintaining agitation of the mixture until it reached a temperature below 30° C. This resulted in Suspension A.

Subsequently, a solution was prepared by mixing 450 g of low-sodium silica sol (34 mass % SiO₂ in water, 200 ppm Na) and 4.5 g of H3PO4 (85 mass %) under agitation at ambient temperature for a period of 30 minutes. This resulted in Suspension B.

The Suspensions A and B were then mixed together with 120 ml of demineralized water added. Thereafter, the solution was agitated for a period of one hour and spray-dried. The spray-dried solid was dried at 200° C. for a period of 16 hours and washed with water at ambient temperature for a period of 2 hours, followed by filtration, drying and calcinations at 700° C. for a period of 2 hours.

### Implementation of Step (a) Conversion

The catalyst tests were carried out on 2 g (35 mesh to 45 mesh particles) of catalyst with a feedstock of essentially pure methanol, at Tinjection=550° C., pressure at 0.5 barg, and hourly space velocity (WHSV=1.6 h⁻¹), in a downflow stainless steel fixed-bed reactor.

Prior to catalytic testing, all catalysts were activated in a stream of N2 (5 Nl/h) until reaching the reaction temperature. Analysis of the products was carried out on-line using a gas chromatograph equipped with a capillary column. The catalytic performance results for the catalyst in Table 1 are reported on carbon, on a dry basis and on a coke-free basis. The results are provided for the average performance of the catalyst during the first 4 hours of operation.

TABLE 1

| WHSV | H − 1 | 1.6 | 1.6 | 4 | 1.6 | 1.6 | 1.6 | 1.6 |
|---|---|---|---|---|---|---|---|---|
| Conversion of | % massC | 100 | 100 | 100 | 98 | 100 | 100 | 100 |
| MeOH | | | | | | | | |
| CH4 | % massC | 2.3 | 1.6 | 1.6 | 3.4 | 1.3 | 2.1 | 1.5 |
| Paraffins | % massC | 6.2 | 6.7 | 5.7 | 8.8 | 8.2 | 8.2 | 8.8 |
| Olefins | % massC | 86 | 85.4 | 87.5 | 79.1 | 83.6 | 82.8 | 82.3 |
| Dienes | % massC | 1 | 0.5 | 0.7 | 1.2 | 0.8 | 0.9 | 0.6 |
| Aromatics | % massC | 6.6 | 7.4 | 6.1 | 10.7 | 6.9 | 8 | 7.6 |
| Ethylene | % massC | 9.8 | 13.9 | 10 | 6.1 | 15.4 | 12.1 | 15.7 |
| Propylene | % massC | 41.4 | 41.8 | 43.3 | 35.4 | 39.6 | 39.2 | 38.4 |
| C4+ Olefins | % massC | 34.8 | 29.7 | 34.2 | 37.6 | 28.6 | 31.5 | 28.3 |
| Catalyst of the | | 1 | 2 | 2 | 3 | 4 | 5 | 6 |
| Example | | | | | | | | |

### Step (a) Performance Results—550° C. −0.5 barg

### Joint Steps (c) Oligomerization and (d) Alkylation, and Step (f) of Hydrogenation

### Properties of the Feedstock Used to Implement Steps (c) and (d)

The properties of the feedstock used are as follows:

TABLE 2

| | Feedstock Properties |
|---|---|
| Density at 15° C. (g/mL) | 0.7251 |
| Bromine Number (g Br/100 g) | 75 |

Distillation Range

| | |
|---|---|
| PI (° C.) | 38.8 |
| T50 (° C.) | 84.5 |
| T95 (C) | 160.7 |
| FBP (° C.) | 164.1 |

The detailed composition of the Feedstock was determined by the GC method.

TABLE 3

| | Distribution of | | Olefin |
|---|---|---|---|
| (% mass) | Hydrocarbons | (mass %) | Composition |
| n-paraffins | 7.1 | C4= | 0.3 |
| i-paraffins | 25.7 | C5= | 15.7 |
| Naphthenes | 11.3 | C6= | 10.1 |
| n-olefins | 10.6 | C7= | 7.6 |
| i-olefins | 19.6 | C8= | 3.5 |
| c-Olefins | 7.9 | C9= | 0.7 |
| Aromatics | 17.7 | C10= | 0.2 |
| | | C11= | 0.1 |
| | | Total Olefins | 38.1 |

### Oligomerization and Alkylation

100 mL of amorphous silica-alumina (ASA) catalyst diluted with 100 mL of inert material (SiC 0.21 mm) was fed into a fixed-bed tubular reactor with an 18 mm internal diameter. Prior to testing, the catalyst was activated at 250° C. (10° C./h) under 135NL/h nitrogen for a period of 8 hours. The temperature was then lowered to 40° C. at the start of the testing program.

100 mL of catalyst based on ZSM-5 (80 mass % MFI with a silica-alumina ratio from 80% to 20 mass % alumina binder) diluted with 100 mL of inert material (SiC 0.21 mm) was fed into a fixed-bed tubular reactor with an internal diameter of 18 mm. Prior to testing, the catalyst was activated at 400° C. (60° C./h) under 160NL/h nitrogen for a period of 2 hours. The temperature was then lowered to 40° C. at the start of the testing program.

### Hydrogenation

Fractionation was carried out on the oligomerization product to recover the 145+ and 165° C.+ oligomerized cuts, which were hydrotreated over a NiMo catalyst. The following operating conditions were chosen: 80 barg, a liquid hourly space velocity LHSV of 1 h−1, H2/hydrocarbon volume ratio of 500 NL/L, in a single pass without recycle, and the temperature was increased from 250° C. to 270° C.

### Example 1—Performance Results Obtained with a Zeolite-Based Catalyst

The feedstock was treated under the following operating conditions: 55 barg, liquid hourly space velocity LHSV of 1 h−1 and at temperatures ranging from 240° C. to 280° C.

TABLE 4

| Yield Structure (% mass) | 240° C. | 260° C. | 280° C. |
|---|---|---|---|
| Yield 145-245° C. | 23 | 27 | 25 |
| Yield 245+ | 5 | 7 | 13 |

The conversion of light olefins (C4-C8 olefins) varies from 65% by mass at 240° C. to 91% by mass at 280° C. The C9+ olefins are not taken into account in the conversion calculation, as they may result from the oligomerization of the light olefins (C4 and C5) present in the feedstock. At 240° C., the conversion of C5-C7 olefins is greater than 88% by mass.

The Olefins may react either by oligomerization or by alkylation with aromatic compounds. It has been observed that the conversion of aromatics varies from 10% by mass at 240° C. to 26% by mass at 280° C. (see Table 5 below). Aromatics are indeed present in the 170-FBP fractions, which indicates that alkylation is indeed taking place.

TABLE 5

| | Feedstock | 240° C. | 260° C. | 280° C. |
|---|---|---|---|---|
| Conversion of IBP-170° C. | — | 10 | 14 | 26 |
| aromatics (mass %) | | | | |
| Aromatic concentration in | — | 3.7 | 5.5 | 11.4 |
| the 170-FBP (mass %) | | | | |

### Example 2—Performance Results Obtained with an Amorphous Silica-Alumina (ASA) Catalyst

The feedstock was treated under the following operating conditions: 25 barg, liquid hourly space velocity LHSV of 1 h⁻¹ and temperatures ranging from 180° C. to 220° C.

TABLE 6

| Yield Structure (% mass) | 180° C. | 200° C. | 220° C. |
|---|---|---|---|
| Yield 145-245° C. | 19 | 20 | 20 |
| 245+ Yield | 12 | 18 | 18 |

The conversion of light olefins (C4-C8 olefins) varies from 80% by mass at 180° C. to nearly 100° C. at 220° C. The C9+ olefins are not taken into account in the conversion calculation, as they may result from the oligomerization of the light olefins (C4 and C5) present in the feedstock. At 180° C., the conversion of C5-C7 olefins is greater than 80% by mass.

The Olefins may react either by oligomerization or by alkylation with aromatic compounds. It has been observed that the conversion of aromatics varies from 28% by mass at 180° C. to 33% by mass at 220° C. (see Table 7 below). Aromatics are indeed present in the FBP at 170° C. fractions, which indicates that alkylation is indeed taking place.

TABLE 7

| | Feedstock | 180° C. | 200° C. | 220° C. |
|---|---|---|---|---|
| Conversion of IBP-170° C. | — | 28 | 31 | 33 |
| aromatics (mass %) | | | | |
| Aromatic concentration in | — | 13.3 | 18.5 | 23.3 |
| the 170-FBP (mass %) | | | | |

The 145+ cuts were hydrotreated using a NiMo catalyst under the conditions described here above. The properties of the hydrogenated cut are described in Table 8 and the detailed composition is summarized in Table 9.

TABLE 8

| | | | ASA (200° C.) | ZEOLITE (240° C.) | ZEOLITE (280° C.) |
|---|---|---|---|---|---|
| | | | Cut 145-245 after | Cut 145-245 after | Cut 145-245 after |
| | Unit | Method | hydrotreating | hydrotreating | hydrotreating |
| Density @ 15° C. | kg/m3 | NF EN ISO | 809.23 | 775.29 | 797.06 |
| | | 12185 | | | |
| Solidification point | ° C. | ASTM D7153 | <−100 | <−100 | <−100 |
| ABEL Flash Point | ° C. | IP170 | | 47.0 | |
| Cetane Calculated | — | ISO4264 | 39.4 | 53.6 | 43.2 |
| Cetane Measured | | | 23.1 | 38.6 | 31.4 |
| Distillation ISO | | ISO3405 | | | |
| IBP | ° C. | | 166 | 165 | 167.5 |
| 5% | ° C. | | 174.5 | 174.3 | 174 |
| 50% | ° C. | | 193 | 188.2 | 189 |
| 95% | ° C. | | 226 | 222.8 | 2242 |
| FBP | ° C. | | 231.5 | 230.6 | 2305 |

TABLE 9

| Cut 145-245 (mass %) | Zeolite 280° C. | ASA 200° C. |
|---|---|---|
| Paraffins | 50.18 | 42.29 |
| Naphthenes | 27.11 | 28.85 |
| DiNaphthenes | 6.58 | 5.01 |
| Alkylaromatics | 14.49 | 22.38 |
| Monoaromatic naphthenol | 1.52 | 1.35 |

Composition of Effluents as Determined by GC×GC

### Additional Example of Implementation of Step (a) Conversion Example of Catalyst

75 g of sample A was introduced into a solution containing 14.25 g of 85 mass % H3PO4 and 225 ml of demineralized water. The suspension was agitated under reflux for a period of 4 hours.

Then 4.9 g of CaCO3 was added to the suspension. Heating of the solution was stopped, while maintaining agitation of the mixture until it reached a temperature below 3000. This resulted in Suspension 51.

Subsequently, a solution was prepared by mixing 450 g of low-sodium silica Sol (34 mass % SiO2 in water, 200 ppm Na) and 4.5 g of H3PO4 (85 mass %) under agitation at ambient temperature for a period of 30 minutes. This resulted in Suspension S2.

The Suspensions S1 and S2 were then mixed together to form a solution. Thereafter, the solution was agitated for a period of one hour and spray-dried, thereby resulting in Catalyst X.

### Implementation of Step (a) Conversion

Tests 1 to 3 described below were carried out in a fixed bed passivated with ceramic, fed with 1.31 g of Catalyst X described above in admixture with SiC. The catalytic bed was maintained in place by means of quartz wool. Alcohol is introduced at the top of the reactor at a flow rate of 0.05 mol Methanol/h/g_{catalyst}. A nitrogen dilution may be added. In the latter case, the alcohol partial pressure is different from the total pressure, as shown in the tables below.

The total pressure was varied (1.3 bara, 5 bara and 10 bara), as was the temperature (450° C., 500° C., 550° C.).

Prior to catalytic testing, the catalyst was activated under N2 (5 Nl/h) until reaching the reaction temperature.

The analysis of the products was carried out using a combination of on-line analysis via gas-phase micro-chromatography of the stream output from the reactor; and off-line analysis by gas-phase chromatography coupled with a flame ionization detector (GC-FID) of the liquid generated by the reaction. The micro-gas chromatograph used has four modules:
Module 1 equipped with a Molecular Sieve capillary column for separating O2, N2, H2, CO and CH4;
Module 2 equipped with a polystyrene-divinylbenzene grafted capillary column (“Plot Q”) for separating MeOH, dimethyl ether (DME), C1 to C3 hydrocarbons, and CO2;
Module 3 equipped with an alumina capillary column for separating C2 to C5 hydrocarbons;
Module 4 equipped with a fused silica type column or “Stabilwax” column for separating water and C6+ hydrocarbons

The results are illustrated in the table below which provides the mass percentage selectivity for a methanol stream:

TABLE 10

| Test | 1 | 2 | 3 |
|---|---|---|---|
| Temperature (° C.) | 500 | 500 | 500 |
| Total Pressure (bara) | 1.3 | 5 | 10 |
| Methanol Partial | 1.04 | 4 | 8 |
| Pressure (bara) | | | |
| Methanol Molar Flux | 0.05 | 0.05 | 0.05 |
| (mol MeOH/h/g | | | |
| catalyst) | | | |
| CH4 selectivity | 0.35 | 0.56 | 0.83 |
| (mass %) | | | |
| C2 Olefin selectivity | 8.98 | 6.07 | 3.96 |
| (mass %) | | | |
| C3 Olefin selectivity | 32.03 | 13.90 | 8.64 |
| (mass %) | | | |
| C4+ Olefin selectivity | 36.97 | 25.48 | 16.91 |
| (mass %) | | | |
| Paraffin selectivity | 15.10 | 34.81 | 18.83 |
| (mass %) | | | |
| Aromatics Selectivity | 6.49 | 18.83 | 22.87 |
| (mass %) | | | |
| Oxygenated | 0.26 | 0.04 | 0.33 |
| compound content | | | |
| (mass %) | | | |
| Ratio of the mass of | 0.8848 | 0.8664 | 0.8658 |
| C3+ olefins to the | | | |
| total mass of olefins | | | |

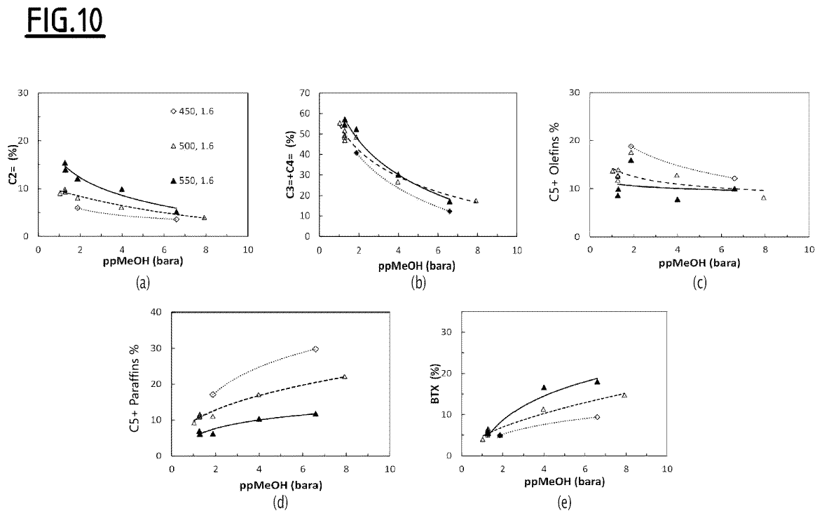

Graphs (a) to (e) shown in FIG. 10 demonstrate the impact of alcohol partial pressure and temperature on selectivities for: ethylene (graph (a)), propylene, and butene (graph (b)), C5+ olefins (graph (c)), C5+ paraffins (graph (d)), and the aromatics benzene, toluene, xylene (graph (e)).

In each graph, the hourly space velocity of methanol relative to the mass of the catalyst WHSV(MeOH) is 1.6/h. The solid triangles correspond to a temperature of 550° C.; the empty triangles correspond to a temperature of 500° C.; and the empty diamonds correspond to a temperature of 450° C.

These examples illustrate that in the above-mentioned temperature range, in particular from 300° C. to 600° C., in particular between 330° C. and 550° C., in particular between 350° C. and 500° C., or between 410° C. and 580° C., with a methanol partial pressure within the above-mentioned range, in particular the range from 100 kPa to 5 MPa, preferably 100 kPa to around 1.0 MPa, with a phosphorus-modified zeolite catalyst, a mixture of paraffins, olefins, aromatics, and water is produced according to the invention in conversion step (a), the ratio of the mass of C3+ olefins to the total mass of olefins being greater than or equal to 0.8.

The same applies when starting with an ethanol stream, as illustrated by the three tests 1E, 1F and 1G below.

The test conditions and selectivity results are as follows:

TABLE 11

| Test | 1E | 1F | 1G |
|---|---|---|---|
| Temperature (° C.) | 500 | 500 | 500 |
| Total Pressure (bara) | 10 | 5 | 3 |
| Ethanol Partial | 8 | 5 | 3 |
| Pressure (bara) | | | |
| Ethanol Molar Flux | 0.05 | 0.125 | 0.03 |
| (mol EtOH/h/g | | | |
| catalyst) | | | |
| CH4 selectivity | 0.19 | 0.13 | 0.24 |
| (mass %) | | | |
| C2 Olefin selectivity | 2.8 | 4.92 | 7.18 |
| (mass %) | | | |
| C3 Olefin selectivity | 7.3 | 10.89 | 12.91 |
| (mass %) | | | |
| C4+ Olefin selectivity | 18 | 23.01 | 17.21 |
| (mass %) | | | |
| Paraffin selectivity | 48.2 | 40.87 | 37.59 |
| (mass %) | | | |
| Aromatics selectivity | 22.8 | 19.97 | 24.8 |
| (mass %) | | | |
| Oxygenated | 0.00 | 0.004 | 0 |
| compound content | | | |
| (mass %) | | | |
| Ratio of the mass of | 0.900 | 0.8732 | 0.8075 |
| C3+ olefins to the | | | |
| total mass of olefins | | | |

In one variant, a 1H test was also carried out in a fluidized bed on 7.2 g of catalyst X with a stream of methanol to be converted. To ensure adequate fluidization properties, the nitrogen flow rate was set at 23.8 mL/min, and alcohol was co-injected at the bottom of the reactor.

The test conditions and selectivity results are as follows:

TABLE 12

| Test | 1H |
|---|---|
| Temperature (° C.) | 500 |
| Total Pressure (bara) | 2.2 |
| Methanol Partial | 1.89 |
| Pressure (bara) | |
| Methanol Molar Flux | 0.05 |
| (mol MeOH/h/g | |
| catalyst) | |
| CH4 selectivity | 0.45 |
| (mass %) | |
| C2 Olefin selectivity | 8.01 |
| (mass %) | |
| C3 Olefin selectivity | 27.05 |
| (mass %) | |
| C4+ Olefin selectivity | 38.93 |
| (mass %) | |
| Paraffin selectivity | 17.88 |
| (mass %) | |
| Aromatics selectivity | 7.59 |
| (mass %) | |
| Oxygenated | 1.21 |
| compound content | |
| (mass %) | |
| Ratio of the mass of | 0.892 |
| C3+ olefins to the | |
| total mass of olefins | |

Prior to the catalytic test, the catalyst was activated under N2 (5 Nl/h) until reaching the reaction temperature.

### Additional Example of Implementation of the Oligomerization Step

On the one hand, 40 mL of ZSM-5 catalyst (80 mass % MFI and 20% alumina binder) sized from 2 mm to 4 mm, diluted with 40 mL of inert material (SiC sized from 1 mm to 1.4 mm) were fed into a fixed-bed tubular reactor with an internal diameter of 16 mm.

Prior to testing, the catalyst was activated at 400° C. (60° C./h) under 160NL/h nitrogen for a period of 2 hours. Thereafter, the temperature was lowered to 40° C. before introducing the feedstock and raising the temperature back to test conditions.

On the other hand, 40 mL of BEA-based catalyst (80 mass % BEA and 20% alumina binder) sized 2-4 mm and diluted with 40 mL of inert material (SiC 0.21 mm) were fed into a fixed-bed tubular reactor with internal diameter of 16 mm.

Prior to testing, the catalyst was activated at 400° C. (60° C./h) under 160NL/h nitrogen for a period of 2 hours. Thereafter, the temperature was lowered to 40° C. before introducing the feedstock and raising the temperature back to test conditions.

The composition of the feedstock used for testing the two catalysts at 55 barg is as follows:

TABLE 13

| Composition of Feedstock | mass % |
|---|---|
| C3 | 25.8 |
| C4 | 14.08 |
| iC4 | 4.5 |
| 1C4 | 2.1 |
| t2C4 | 4.5 |
| c2C4 | 2.96 |
| 1C5 | 4.7 |
| 1C6 | 2.3 |
| BTX | 4.98 |
| Benzene | 0.75 |
| Toluene | 1.49 |
| Xylene | 2.74 |
| nC7 | 48.1 |

Yield structures are estimated on the basis of the following cut points
Light Olefins: IBP-80° C.
Naphta: 80° C.-145° C.
Jet fuel: 145° C.-300° C.
Diesel: >300° C.

### Example Oligo-1—Performance Results Obtained with ZSM-5 Catalyst

The feedstock was treated and processed under the following operating conditions: 55 barg, liquid hourly space velocity LHSV of 1 h⁻¹ and temperatures ranging from 180° C. to 240° C.

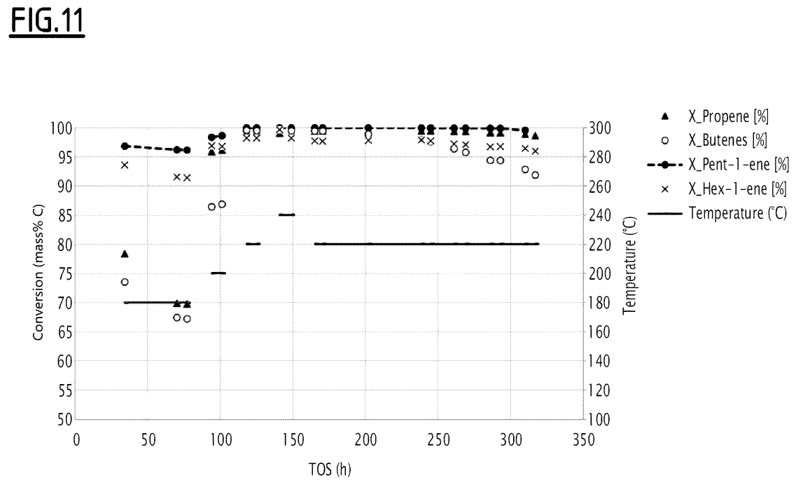

The conversion of light olefins (C3 to C6 olefins) is shown in FIG. 11. It is above 90% by mass for temperatures above 220° C. At 220° C., the conversion of butenes and hexenes decreases with time.

In terms of yield structure, once the contribution of the diluent (n-heptane in this case) has been subtracted, yields at 220° C. are as follows:

TABLE 14

| | Yield at 220° C. |
|---|---|
| Time of Stream (TOS, h) | 165 |
| Light Olefins: IBP-80° C. | 3.5 |
| Naphtha: 80-145° C. | 33.2 |
| Jet fuel: 145° C.-300° C. | 55.2 |
| Diesel: >300° C. | 6.4 |

### Example Oligo-2—Performance Results Obtained with the Bea-Based Catalyst

The feedstock was treated and processed under the following operating conditions: 55 barg, liquid hourly space velocity LHSV of 1/h and at temperatures ranging from 150° C. to 220° C.

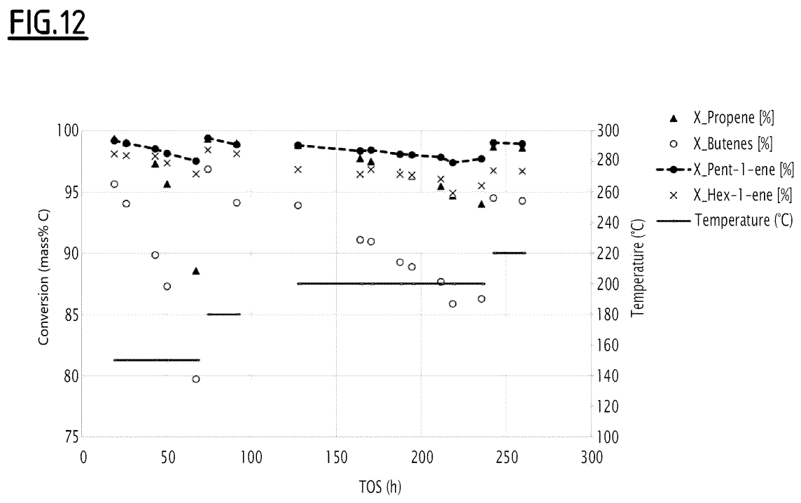

The conversion of C5 and C6 light olefins is shown in FIG. 12. It is above 90% by mass for temperatures from 200° C. upwards.

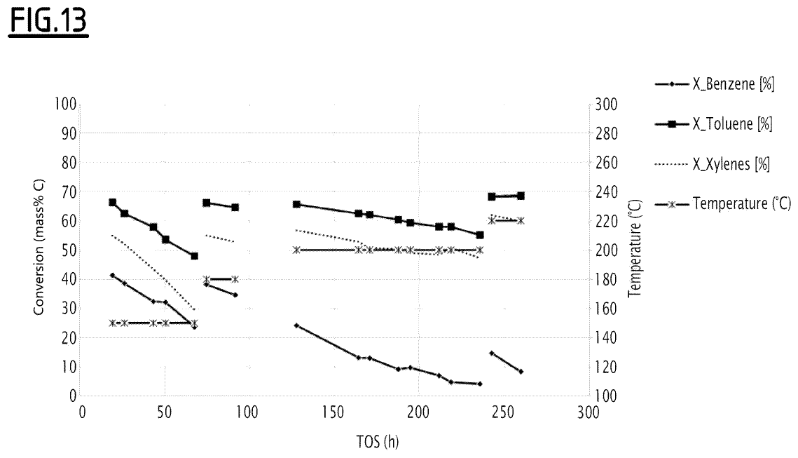

The olefins may react either by oligomerization or by alkylation with aromatic compounds. The aromatic compounds are partially converted and at iso-temperatures, with the degree of conversion of these aromatics decreasing over time, as illustrated in FIG. 13.

In terms of yield structure, once the contribution of the diluent (n-heptane in this case) has been subtracted, the yields at 200° C. are as follows:

TABLE 15

| | Yield at 200° C. |
|---|---|
| Time of Stream (TOS, h) | 127 |
| Light Olefins: IBP-80° C. | 8.6 |
| Naphtha: 80-145° C. | 30.9 |
| Jet fuel: 145° C.-300° C. | 53.3 |
| Diesel: >300° C. | 4.4 |

These results show that for the oligomerization and alkylation conditions defined above, in particular: for temperatures from 150° C. to 400° C., preferably from 180° C. to 350° C., even more preferably from 180° C. to 290° C.; for a weight hourly space velocity, WHSV) of the feed, for example from 0.1 h⁻¹ to 20 h⁻¹, preferably from 0.5 h⁻¹ to 10 h⁻¹, even more preferably from 0.8 h⁻¹ to 5 h⁻¹; with zeolite-type catalysts, it is possible to very efficiently convert a feedstock obtained at the conclusion of step (a) into a significant quantity of jet fuel.

1. A hydrocarbon producing process, comprising:
(a) converting a C1 to C6 alcohol stream to produce a mixture containing paraffins, olefins, aromatics, and water;
(b) separating water from the mixture containing paraffins, olefins, aromatics, and water to form a water-depleted mixture;
the water-depleted mixture being separated and/or treated to recover hydrocarbons;
the process comprising adding, in the converting of the C1 to C6 alcohol stream, a carbon dioxide-containing stream, and jointly converting carbon dioxide to carbon monoxide in the converting of the C1-C6 alcohol stream.

2. A process according to claim 1, wherein the carbon dioxide-containing stream comprises more than 5% by mass of carbon dioxide.

3. A process according to claim 1, wherein a mass ratio of carbon dioxide to C1-C6 alcohols in the C1 to C6 alcohol stream supplied to the converting of the C1 to C6 alcohol stream is between 5% and 75%.

4. A process according to claim 1, wherein at least 2 mol % of the carbon dioxide contained in the carbon dioxide-containing stream is converted in carbon monoxide during the converting of the C1 to C6 alcohol stream.

5. A process according to claim 1, wherein the converting of the C1 to C6 alcohol stream is carried out using at least one catalyst comprising molecular sieves containing at least 10 oxygen atom sized pores (10-MR) or larger in a microporous structure of the molecular sieves.

6. A process according to claim 5, wherein the at least one catalyst comprises: a phosphorus-modified zeolite, a composite catalyst comprising at least 0.1% by mass of silicate; or a molecular sieve modified with phosphorus (P) and with an alkaline-earth or rare-earth metal (M).

7. A process according to claim 5, wherein the at least one catalyst has been modified by adding one or more metals selected from: Group IIB metals; Group IIIB metals; Group VIIIB transition metals; Group VIB metals; Group IB metals; or metals from the lanthanide group.

8. A process according to claim 5, wherein the at least one catalyst is a phosphorus-modified zeolite having partially an ALPO structure, or is a B-modified zeolite.

9. A process according to claim 1, comprising separating the water-depleted mixture in a C1-C2 hydrocarbon fraction, and in a C3+ hydrocarbon fraction.

10. A process according to claim 9, wherein carbon dioxide, carbon monoxide and hydrogen present in the C1-C2 hydrocarbon fraction are separated from hydrocarbons present in the C1-C2 hydrocarbon fraction.

11. A process according to claim 1, comprising:
(c) oligomerizing olefins from the water-depleted mixture to form oligomerized olefins;
(d) alkylating aromatics from the water-depleted mixture to form alkylated aromatics;
(e) forming a hydrocarbon stream from at least a portion the oligomerized olefins and at least a portion of the alkylated aromatics.

12. A process according to claim 11, comprising:
(f) hydrogenating the hydrocarbon stream to form a hydrogenated hydrocarbon stream;
(g) recovering at least one jet fuel fraction from the hydrogenated hydrocarbon stream.

13. A process according to claim 12, wherein the at least one jet fuel fraction comprises between 2% by volume and 30% by volume of C8+ aromatics.

14. A process according to claim 12, where, in the mixture containing paraffins, olefins, aromatics, and water, a ratio of a mass of C3+ olefins to a total mass of olefins is greater than or equal to 0.8.

15. A hydrocarbon producing plant, comprising:
a E conversion stage configured to convert a C1 to C6 alcohol stream to produce a mixture containing paraffins, olefins, aromatics, and water;
a separation stage configured to separate the water from the mixture containing paraffins, olefins, aromatics, and water to form a water-depleted mixture;
at least one separation/treatment stage configured to separate and/or treat the water-depleted mixture to recover hydrocarbons,
the conversion stage comprising at least one supply conduit configured to supply a carbon dioxide-containing stream, the conversion stage being configured to convert carbon dioxide arising from the carbon dioxide-containing stream in carbon monoxide jointly with the conversion of the C1 to C6 alcohol stream.
